(19) United States
(12) Patent Application Publication
(10) Pub. No.: US 2023/0372460 A1
(43) Pub. Date: Nov. 23, 2023

(54) METHODS OF TREATING CANCER AND MONITORING ANTI-CANCER IMMUNITY

(71) Applicant: LURONG ZHANG, BETHESDA, MD (US)

(72) Inventor: LURONG ZHANG, BETHESDA, MD (US)

(21) Appl. No.: 18/081,178

(22) Filed: Dec. 14, 2022

Related U.S. Application Data
(60) Provisional application No. 63/289,295, filed on Dec. 14, 2021.

(51) Int. Cl. A61K 39/00; A61P 35/00; C12N 5/09; C12N 13/00; G01N 33/68; G01N 33/574

(52) U.S. Cl. CPC A61K 39/0011; A61P 35/00; C12N 5/0693; C12N 13/00; G01N 33/6854; G01N 33/57484; C12N 2521/00

(57) ABSTRACT

The invention relates to induced neoantigen vaccines and a method of using same to treat cancer by enhancing a patient's anti-cancer immunity. The method involves application of an induction radiation to the patient to generate an “in situ vaccine” in vivo, subsequent removal of the tumor, subjecting its cells to a survival pressure for further production of neoantigens in vitro, and processing of the cells to obtain a self-tumor vaccine. The invention provides comprehensive mobilization of individualized anti-cancer active immunity via sequential combination of means of cancer treatments (e.g., radiotherapy, surgery, chemotherapy). Another aspect of the invention relates to an immunoassay protocol to monitor parameters indicative of the cellular and humoral anti-cancer immunity of a patient.

### CROSS-REFERENCE TO A RELATED APPLICATION

This application claims priority to U.S. provisional patent application No. 63/289,295, filed Dec. 14, 2021, the entire contents of which are incorporated herein by reference.

### BACKGROUND OF THE INVENTION

Cancer is a leading cause of death worldwide, which is typically treated with four major means: surgery, chemotherapy, radiotherapy, and immunotherapy. Oncologists regularly face the challenge of how to sequentially integrate these four treatment methods to more effectively treat cancer patients. Furthermore, despite the improvements that have been made over decades, there still are downsides to these methods that need to be addressed.

Surgical operation can be the bane of metastases due to its extrusion of cancer cells into the patient's circulation that can survive and grow in other sites of the body away from the tumor being removed. Surgical trauma can also induce local and systemic inflammatory responses that may contribute to the accelerated growth of residual cancer or metastases. Therefore, it is worthy to explore how anti-cancer immunity can be built up before surgery to kill the cancer cells extruded into the blood during surgery and reduce the risk of post-operation metastasis.

Chemotherapy (CT) systemically kills cancer cells no matter where and how small/few they are. Therefore, it is one of major anti-cancer treatments. However, CT damages normal cells along with cancer cells, especially the drug-sensitive immune cells. CT further exacerbates the host immunity by generating side effects such as anorexia, nausea, and vomiting. Similarly, if radiotherapy is combined with chemotherapy, the body's capacity to build the active anti-cancer immunity is also compromised, as cytotoxic drugs kill drug-sensitive immune cells. Furthermore, CT can facilitate tumor cells' evasion of the body's immune surveillance as it can induce rapid mutations in tumor cells that result in selective growth of drug-resistant “worst tumor cells.” When and how to integrate CT into a whole cancer treatment process need to be considered.

Targeted anti-cancer drug is a more recently emerged and new form of chemotherapy with less toxicity due to its targeting of the growth receptors and/or signal pathways that exist in normal cells but are upregulated or uncontrolled in cancer cells. Although targeted drugs can be successful in suspending tumor growth for some period, they rarely cure cancer. Tumors eventually develop drug resistance and resume growing. While new targeted drugs continue to be developed, it is a challenge for physicians to determine when and which drug to use for an effective treatment. It should be pointed out that tumor cells killed by targeted drugs can release antigens in vivo and trigger an anti-cancer immune response to a certain degree due to the relatively intact host immunity, unlike conventional cytotoxic CT that greatly damages the whole immune system.

Another relatively new treatment approach is immunotherapy, which comes in two forms: passive immunotherapy and active immunotherapy.

Passive immunotherapy is achieved by an infusion of antibodies or cells. The antibodies include targeted antibodies (such as anti-CD19/CD20, anti-HER2/EGFR, anti-VEGF/VEGFR, etc.) and checkpoint inhibitors (such as anti-PD-1/PD-L1, etc.) that block the interaction of receptors and ligands on cell surfaces and inhibit downstream signals. Mostly, they target tumor-associated antigens (TAAs) as opposed to tumor-specific antigens (TSAs). TAAs are found at elevated levels on tumor cells but also expressed at lower levels on healthy cells. Thus, these antibodies can cause intolerable side effects as they interfere with functions of normal cells. Furthermore, tumor cells can develop drug-resistance even after a period of regression, similar to targeted drugs that aim at certain signaling pathways, causing the cancer to bounce back. On the other hand, cells in adoptive cell transfer therapies have short half-lives, have difficulty penetrating solid tumors, and do not build sufficient force to kill rapidly proliferating cancer cells.

Active immunotherapy involves antigens (i.e., “vaccines”) to set an anti-cancer immune response in patients to fight cancer cells. One type of such vaccines is derived from cancer-related pathogens such as Human papillomavirus (HPV), Epstein-Barr virus (EBV), hepatitis B virus (HBV), and Helicobacter pylori and so on that can induce cancer. Immunization with these vaccines can prevent cancer formation. Another type of active immunotherapy vaccine is derived from neoantigens, i.e., TSAs, which are new proteins formed in cancer cells when certain mutations occur in tumor DNA. While neoantigen-based immunotherapy has great potential to be an effective treatment and prevention of cancer, the existing approach suffers from multiple shortcomings and thus faces several challenges.

First, there is still a need to induce production of neoantigens that can re-arouse the host's anti-cancer immunity. The current method directly uses the tumor obtained via surgery or biopsy to define neoantigens. As TSAs are difficult to define, technologies such as Next-Generation Sequencing (NGS) are used to screen for neoantigens in isolated tumors by identifying mutant differences between the normal cells and cancer cells. However, tumors that are being used for neoantigen identification have already had a chance to grow in a patient's body because they (and their existing antigens) have escaped the body's immune surveillance. Thus, the neoantigens found on such tumors might have a weak immunogenicity and would not serve as an effective basis for a vaccine.

There is also a need to obtain a whole set of neoantigens to trigger the host's anti-cancer immunity. According to the two-hit hypothesis, a set (that ranges from a few dozen to hundreds) of mutations can cause the formation, progression and/or treatment-resistance of cancer. Thus, multiple neoantigens might be necessary to effectuate a desired result. With the existing method, it is difficult to identify and synthesize the complete set of neoantigens necessary for activating the anti-cancer immunity in a patient.

Obviously, there is a need to minimize/eliminate uncertainties in the preparation of anti-cancer immunotherapy. For the manufacture of neoantigen-based vaccines generally, the identified neoantigen peptides are synthesized and undergo a selection process, e.g., human leukocyte antigens (HLA) matching, to ensure a safe presentation of the neoantigens to a patient. The currently existing method is a time-consuming and labor-intensive process, involving identification of neoantigens using NGS, synthesis of neoantigen peptides, and HLA matching, which takes about 3 months or longer. In addition, as stated previously, these antigens obtained directly from tumors have already developed by “escaping or paralyzing” the host immune system. Therefore, they likely will be tolerated by the body as weak antigens. Furthermore, with each passage of time, a vaccine may become ineffective against target tumors especially if the tumors are undergoing rapid growth and mutations.

Thus, autologous tumor vaccines (vaccines produced from cells obtained from the same patient to be treated) are preferable. Furthermore, due to a formed tumor having already escaped or paralyzed the patient's immunity, a new method that can enhance the expression of neoantigens in an existing tumor is needed to arouse the patient's immunity. In addition, the tumor vaccines should be made relatively faster, safer, and cheaper than conventional vaccines. This requires new methods to make autologous tumor vaccines utilizing tumors either grown in vivo or surgically obtained in vitro.

Radiotherapy (RT), like surgery, is a local treatment with high energy radiation to break the DNA and cause cancer cell death without harming normal cells outside the radiation field, especially the bone marrow and other immune organs. Therefore, RT is a good means to induce a production of vaccine and trigger anti-cancer immunity.

Taken together, surgery, chemotherapy, radiotherapy and immunotherapy have been used as four major means for cancer treatment, however, how to integrate these four means in a novel way to achieve the best benefits for patients is explored in this invention.

Lastly, there is a need for a set of immunological method to systematically monitor changes in a patient's anti-cancer immunity at cellular and humoral levels before, during, and/or after a cancer treatment. The treatment can be vaccine administration, radiotherapy, conventional chemotherapy, targeted-drug therapy, antibody/cell therapy, and even PD-1 inhibition treatment. It is certain that upon tumor cell death due to any treatment method, antigens are commonly released that may be subsequently processed to generate an “in situ vaccine,” triggering the host's anti-cancer immunity to clean up the dead cells and shrink the remaining tumor. Currently, no routine assay is available as an “eye” for physicians to “see and monitor” the anti-cancer immunity being induced by a treatment. Every cancer patient exhibits a different degree of immunological parameters that can be detected even if the patient's immune system is not strong enough to eradicate cancer. Monitoring the anti-cancer immunity of individual patients before, during, and/or after a treatment is beneficial for understanding the patients' immune response to a given treatment and tailoring the treatment methods.

### BRIEF SUMMARY OF THE INVENTION

In one aspect, the invention provides comprehensive mobilization of individualized anti-cancer active immunity via sequential combination of means of cancer treatments (e.g., radiotherapy, surgery, chemotherapy) and monitoring of anti-cancer immunity by a set of parameters.

In one aspect, the invention relates to a method of treating cancer by enhancing a patient's active anti-cancer immunity through a combination strategy involving the use of an induction radiation to produce an “in situ vaccine” in a patient, followed by the removal of a tumor from the patient, the production a “self-tumor vaccine” by culturing the removed tumor cells under a survival pressure to induce production of additional neoantigens, and administration of the self-tumor vaccine as an active immunotherapy at an appropriate time.

In one embodiment, the method of treatment according to the present invention comprises the following steps that can integrate the four means of cancer therapies in a novel way: 1) treating a patient's tumor with a first induction radiation or/and a low-dose half course chemotherapy with or without other treatments such as GM-CSF and/or immunomodulators to create an in-situ vaccine; 2) removing the tumor from the patient; 3) culturing the tumor cells under a survival pressure to prepare a self-tumor vaccine comprising in vitro-induced neoantigens; and 4) intradermally administering the self-tumor vaccine to the patient at an appropriate time, optionally with another cancer treatment such as immunoenhancers and/or immunosuppressive blockers. In further embodiments, if recurrence or oligo-metastases are detected after the fourth step, all four steps of the treatment method may be repeated.

In another aspect, the present invention relates to induced neoantigen vaccines prepared from a patient's own tumor cells and a method of preparation thereof. In some embodiments, the method comprises: (1) stimulating the production of an “in situ vaccine” in a patient and thereby enhancing the anti-cancer immunity of a patient having a cancerous tumor by treating the patient with a first induction radiation and/or a low-dose half course chemotherapy with or without another treatment such as GM-CSF and/or immunomodulators; (2) removing the tumor cells from the patient; (3) culturing the tumor cells under a survival pressure to prepare a self-tumor vaccine comprising in vitro-induced neoantigens.

Another aspect of the present invention is directed to a cell irradiator utilized in preparing the self-tumor vaccine of the present invention. The cell irradiator comprises a high voltage transformer, an X-ray tube, a control system, and an X-ray chamber for irradiating the removed tumor cells with a required dose of X-ray. Preferably, the control system can adjust the radiation dose rate to be about 0.5 Gy/min. to about 2 Gy/min. Preferably, the cell irradiator is capable of delivering from about 6 Gy to about 8 Gy of X-ray per fraction to a cell sample.

A further aspect of the invention is directed to an immunoassay protocol and an immunoassay kit that are used to monitor the changes in the humoral and cellular immunity against cancerous tumors in a patient as a result of cancer treatments, including the treatment method of the present invention. In some embodiments, the immunoassay protocol measures certain biological parameters before and after the commencement of a treatment, wherein the protocol includes assays for one or more CD4⁺IFNγ⁺, NK⁺IFNγ⁺, MDSC, and CD25⁺ cells, an assay for anti-cancer antibody titer, or cytokine assays for IL2, IL6, IFNγ, etc., and a circulating tumor cells (CTC) test. Preferably, the immunoassays are combined with the cancer treatment method according to the present invention to aid in the evaluation of the efficacy of a patient's current treatment as well as in the determination of subsequent therapeutic steps for the patient if necessary.

### BRIEF DESCRIPTION OF THE DRAWINGS

FIG. 1 shows the results of two-dimensional electrophoresis and Western blot analysis on tumor cells that were subjected to an induction radiation.

FIG. 2 shows a summary of the results of proteomic analysis, demonstrating commonly shared new and increased neoantigens between the exosomes and lysates of cultured Hep G2 cells irradiated with 8 Gy once as well as the exosomes of the plasma of hepatoma patients who received a single 8 Gy SBRT induction radiation.

FIG. 3 shows graphs comparing the expression of two neoantigens CD151 and KPNA2 identified in FIG. 2 in the hepatoma tissues and in adjacent normal tissues, based on the information obtained from the National Institutes of Health (NIH) real-world databases in The Cancer Genome Atlas (TCGA).

FIG. 4 shows graphs demonstrating the correlation between the up-regulated neoantigens CD151 and KPNA2 identified in FIG. 2 and poor clinical outcome, based on the information obtained from the NIH's real-world databases in The Cancer Genome Atlas (TCGA).

FIG. 5 shows a schematic of an experimental scheme to demonstrate the anti-cancer effectiveness of an induction radiation-induced “in situ vaccine” in ICR mice.

FIG. 6 shows a schematic of an experimental scheme for the effectiveness of an induction radiation-induced “in situ vaccine” on anti-lung metastasis in BLAB/c mice.

FIG. 7 shows results of the experiment depicted in FIG. 6, demonstrating the effects of the induction radiation-induced “in situ vaccine” on the inhibition of experimental lung metastasis.

FIG. 8A shows additional results of the experiment depicted in FIG. 6, demonstrating the effects of the induction radiation-induced “in situ vaccine” on the inhibition of lung metastasis.

FIG. 8B shows additional results of the experiment depicted in FIG. 6, demonstrating the effects of the induction radiation-induced “in situ vaccine” on the survival rate.

FIG. 9A shows a graph comparing the antibody titer of the mice in the experiment depicted in FIG. 6.

FIG. 9B shows a graph comparing the lymphocyte transformation in cultures with or without the 8 Gy-irradiated 4T1 cell lysate as mitogen, wherein the lymphocytes were obtained from a group of mice in the experiment depicted in FIG. 6.

FIG. 10 shows a flowchart as an exemplary embodiment of the immunoassay protocol according to the present invention.

FIG. 11 shows a graph of the results of lymphocyte transformation test demonstrating that about two-thirds (⅔) of the patients having thyroid cancer had an increased anti-cancer lymphocyte transformation after being treated with ¹³¹I internal irradiation.

FIG. 12 shows a graph of the titer of anti-cancer antibody induced by the ¹³¹I internal radiation as described in Example 12 was higher in patients with a focal cancer (visible focus, indicated as “Big tumor volume”) than that in patients with no visible lesions (indicated as “Small tumor volume”).

FIG. 13 shows graphs of increased levels of cytokines IL-1b and CXCL-16 induced by ¹³¹I internal radiation of patients with thyroid cancer of Example 12.

FIG. 14 shows MRI and CT scans of a hepatoma patient before and after a single 8 Gy SBRT treatment.

FIG. 15 shows a graph comparing the titer of anti-hepatoma antibodies in the plasma collected from the patient of Example 15 at different dilutions and at different timepoints before and after the 8 Gy SBRT treatment.

FIG. 16 shows graphs of the results of the anti-NPC (Nasopharyngeal Cancer) antibody titer in the plasma of NPC patients at different timepoints before and after a conventional radiotherapy.

FIG. 17 shows a graph and a corresponding table of the dynamic changes of anti-NPC cancer lymphocyte transformation and lymphocyte subsets in after a combination therapy of radiotherapy and chemotherapy.

FIG. 18 shows a schematic of an experimental scheme for the evaluation of the effectiveness of an induced neoantigen vaccine prepared in vitro on primary tumor growth in a mouse model.

FIG. 19 shows graphs of primary tumor growth curves of the mice of Example 22.

FIG. 20A shows a graph of primary tumor growth curves of mice in the control group immunized with a PBS solution only.

FIG. 20B shows a graph of primary tumor growth curves of mice that were immunized with a vaccine derived from 8 Gy radiation-induced neoantigens.

FIG. 21A shows in vivo live imaging results of the effect of the neoantigen-induced vaccine on experimental lung metastasis after active immunization in a mouse model.

FIG. 21B shows a graph of total flux (photons/s) of the three groups of mice with lung metastases corresponding to FIG. 21A.

FIG. 22A shows a graph of HSP70 levels in tumor cells before and after 8 Gy induction radiation.

FIG. 22B shows a graph of antibody titer in the plasma samples of four groups of mice, demonstrating the most increase in the group that was immunized with induced neoantigens of 4T1 cells.

FIG. 23A shows an image of transformed lymphocytes cultured in the absence of 8 Gy-induced 4T1 cell lysate as a mitogen.

FIG. 23B shows an image of transformed lymphocytes cultured in the presence of 8 Gy-induced 4T1 cell lysate as a mitogen.

FIG. 24A shows a graph of results of lymphocyte cytotoxicity assay using 4T1 as target tumor cells and lactate dehydrogenase (LDH) as a cell death indicator.

FIG. 24B shows a table summarizing the results of antibody titer in the plasma of mice immunized with different vaccines.

FIG. 25A shows a graph demonstrating the levels of the lymphocyte subsets CD8 and CD4 in the blood of the mice immunized with various vaccines described in Example 30.

FIG. 25B shows a graph demonstrating the levels of Treg cells in the blood of the mice immunized with various vaccines described in Example 30.

FIG. 26A shows a graph of myeloid-derived suppressor cells (MDSC) levels in the bone marrow of the three groups of mice described in Example 30.

FIG. 26B shows a graph of myeloid-derived suppressor cells (MDSC) levels in the peripheral blood of the three groups of mice described in Example 30.

FIG. 27 is a flow chart depicting an exemplary procedure of the preparation and use of a self-tumor vaccine of the present invention for active immunotherapy.

FIG. 28 shows a graph summarizing and the distribution of patients who received the self-tumor vaccine of the present invention based on the frequency of immunization in the phase I clinical trial as described in Example 33.

FIG. 29 shows a graph summarizing the incidence of fever in the patients immunized with the self-tumor vaccine, observed in the phase I clinical trial of Example 33.

FIG. 30 shows a summary of the systemic and local side effects of the self-tumor vaccine of the present invention, observed in the phase I clinical trial of Example 33.

FIG. 31 shows results of Western blotting using capillary electrophoresis for autoantibodies against self-antigens on the plasma samples collected from four patients before and after immunization a self-tumor vaccine.

FIG. 32 is a table summarizing a panel of autoantibodies that were examined in the plasma of the patients of Example 36, indicating negative results for all of the listed autoantibodies.

FIG. 33 shows a graph demonstrating the effects of different therapies on lymphocyte transformation stimulated by tumor antigens.

FIG. 34 shows graphs of lymphocyte transformation and lymphocyte subsets of patients who received radiotherapy in combination with GM-CSF.

FIG. 35 shows a flowchart of an exemplary embodiment of the cancer treatment method according to the present invention.

FIG. 36 shows a control panel of a cell irradiator according to the present invention.

FIG. 37A shows a photograph of the front view of a cell irradiator according to the present invention.

FIG. 37B shows a photograph of the side view of a cell irradiator according to the present invention connected to a transformer according to the present invention.

FIG. 37C shows a photograph of a control panel according to the present invention connected to a transformer.

FIG. 37D shows a photograph of radiators disposed on top of a transformer according to the present invention.

FIG. 38 shows a table and a corresponding graph of the radiation rates of an embodiment of the cell irradiator of the present invention.

FIG. 39 shows a simplified overview of the cancer treatment method according to the present invention and its effect.

FIG. 40 shows an exemplary overview of an embodiment of the immunoassay protocol evaluating humoral immunity and cellular immunity according to the present invention.

### DETAILED DESCRIPTION OF THE INVENTION

The present invention relates to a method of treating a patient having a cancerous tumor through a comprehensive mobilization of the patient's anti-cancer immunity by utilizing two types of induced neoantigen vaccines: an “in situ vaccine” comprising neoantigens induced in vivo in the patient; and a self-tumor vaccine prepared from surgically removed or biopsied tumor tissues from the patient and induced in vitro. The present invention effectively coordinates surgery, chemotherapy, radiotherapy, and immunotherapy in a novel way to create an individualized in situ vaccine as well as a self-tumor vaccine to maximize the anti-cancer immunity in a patient via comprehensive mobilization of individualized anti-cancer active immunity via sequential combination of means of cancer treatments (e.g., radiotherapy, surgery, chemotherapy).

The invention also relates to a method of making such vaccines and methods of monitoring the patient's anti-cancer immunity. Utilization of the two vaccines is incorporated into the patient's treatment regimen, and can be combined with other conventional cancer treatment methods. Thus, the four major means for anti-cancer can be sequentially integrated.

In some embodiments, the present invention provides a method to prepare the individualized cancer vaccines both in vivo and in vitro, to integrate the four means of anti-cancer treatment to enhance active immunity, and to monitor the alterations of humoral and cellular anti-cancer immunity after treatments.

When cancer cells are irradiated or subjected to a similar stress, to avoid cell death, the cells express a large amount of oncoproteins to help their survival or escape the “death stress.” These oncoproteins are the neoantigens that can wake up the paralyzed host immunity. Therefore, the induction radiotherapy-induced neoantigens act as “in situ vaccine.” Similarly, tumor tissues obtained from a patient by surgery or biopsy can be freshly prepared in a culture and then exposed to radiation or a similar stress to trigger the expression of neoantigens. These neoantigens can then be used to immunize the same patient as a “self-tumor vaccine” comprising the whole set of oncoproteins without the need for HLA matching and a time- or labor consuming preparation of a cancer vaccine to induce active anti-cancer immunity.

The present invention maximizes a patient's anti-cancer immunity by utilizing individualized and comprehensive methods of treatments and monitoring, which can be integrated into a whole process utilizing the four major means of cancer treatment. Some of the advantages of this invention include but are not limited to:
Utilization of an induction radiation or half course chemotherapy to produce an “in situ vaccine” to trigger active anti-cancer immunity before surgery to prevent the cancer cells extruding into the blood from forming metastases;
Relative ease and speed with which the self-tumor vaccines can be produced, with the preparation taking about a few days;
Convenience as a result of eliminating the need to select or purify potentially viable neoantigens for vaccine production;
A potential for creating multivalent tumor vaccines as the self-tumor vaccines according to the present invention to cover a wide range of neoantigens;
Expected safety because the vaccines prepared according to the present invention are autologous;
Versatility of the vaccine which can be utilized at any time ranging from the early stage of cancer through after metastasis or recurrence;
Mitigation of risks of metastases associated with surgeries or during the cancer course, as the “in situ vaccine” and self-tumor vaccine according to the present invention enhance the patient's anti-cancer immunity; and
Adaptability and flexibility of the treatment method due to the autologous nature of the “in situ vaccine” and self-tumor vaccine as well as scheduled monitoring of the patients' immune response to the vaccines.

Furthermore, some notable differences between exemplary prior art methods of making a tumor-based vaccine and the methods according to the present invention are described in Table I below. Exemplary prior art methods are described in references including: Lee K L et al., Efficient Tumor Clearance and Diversified Immunity Through Neoepitope Vaccines and Combinatorial Immunotherapy, Cancer Immunol Res., 7(8), 1359-1370 (2019); Ott P A et al., An Immunogenic Personal Neoantigen Vaccine for Patients with Melanoma, Nature, 547(7662), 217-221 (2017); and Keskin D B et al., Neoantigen Vaccine Generates Intratumoral T Cell Responses in Phase Ib Glioblastoma Trial, Nature, 565(7738), 234-239 (2019).

TABLE 1

| | | Methods According to the |
|---|---|---|
| | Exemplary Prior Art Methods | Present Invention |
| Method for searching | NGS sequencing, which looks | No searching required, and |
| for neoantigens and | for mutations, and takes about | induction of tumor neoantigens |
| required time | 2-4 weeks or more | takes much quicker |
| Compositions of tumor | Mixture of about 20 various | All tumor neoantigens |
| vaccine | neoantigen peptides or co- | |
| | constructed 125 TAA mRNAs | |
| Method for preparing a | Synthesis of HLA-matched 20 | Direct induction of tumor |
| tumor vaccine | neoantigen peptides or | neoantigens which do not |
| | construction of vectors for | require HLA matching because |
| | expression of 125 TAA | they are autologous |
| | mRNAs | |
| Time required, | About 3-4 months, | About 2-3 days, simpler, and |
| complexity, and cost of | complicated, and costly | cheaper |
| making a tumor vaccine | | |
| Types of neoantigens in | May have weak | Can be more readily recognized |
| tumor vaccine | immunogenicity and are not | by T-cells in the host and are |
| | induced | newly induced |

### Individualized Vaccines and a Method of Treating Cancerous Tumors Using Same

One aspect of the present invention is directed to a method of treating a patient having a tumorous cancer. Tumors in the context of the present invention can be a wide range of tumors including a primary tumor or secondary metastatic tumors, as well as oligodendroglioma (“oligotumor”) or multiple tumor mass. Tumors can be derived from various sources including but are not limited to, brain, lung, head and neck, skin, pancreas, gastrointestinal tract, bladder, reproductive tract, spinal cord, spleen, kidney, liver, limbs, bone, tongue, and throat. Tumors also include non-solid tumors such as those derived from blood or bone marrow.

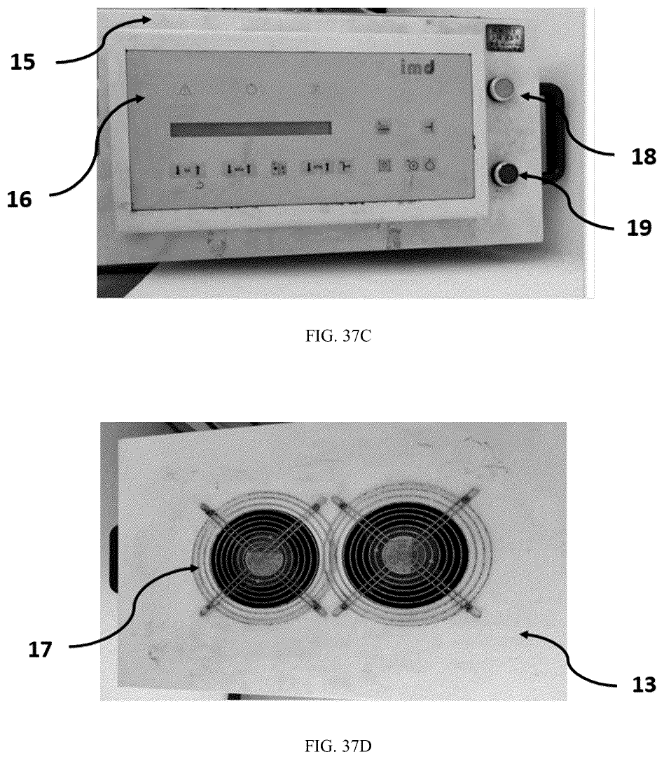

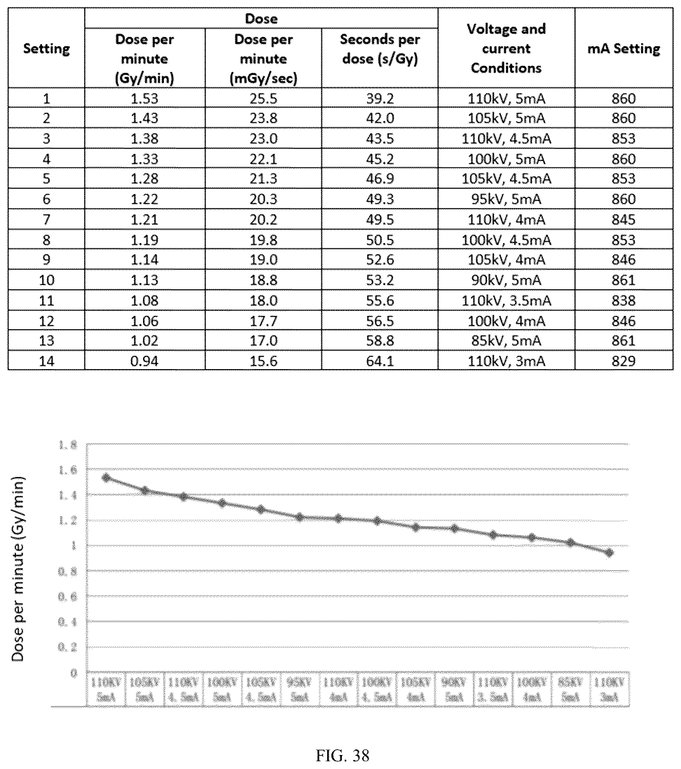

The cancer treatment method according to the present invention comprises four steps and is designed to maximally trigger the systemic anti-cancer immunity of a patient by combining the benefits of an induced “in situ” vaccine with an individualized self-tumor vaccine obtained from the patient's tumor cells that is administered intradermally into four limbs. A simplified overview of the method and its effect is provided in FIG. 38.

First Step

The first step of the cancer treatment method according to the present invention comprises treating a patient having a cancerous tumor with a first induction radiation, a preferred method, to prepare an “in situ vaccine.” Alternatively, or additionally to the first induction radiation, a low-dose half-course chemotherapy can be used with or without GM-CSF and/or immunomodulators. As used herein, low-dose half-course chemotherapy is chemotherapy that is lower in dose, preferably a half or less, than the corresponding chemotherapy in full dose or course. The purpose of the first induction radiation or a low-dose half-course chemotherapy is to induce the tumors to produce neoantigens, i.e., new, tumor-specific proteins that form on cancer cells, so as to create an “in situ vaccine” that triggers an active anti-cancer immune response in the host. “In situ vaccine” refers to an approach that exploits neoantigens formed at the tumor site to induce a neoantigen-specific active immunity. There is no need to identify or isolate the neoantigens, which are formed by the tumor cells in vivo in response to the first induction radiation or a low-dose half-course chemotherapy.

Without being bound by a theory, the inventors of the present invention hypothesized that tumors that have grown in a patient have already escaped or are being tolerated by the host's immunity and thus, additional neoantigens must be induced in order to enhance their immunogenicity. Benefits of this first step include, but are not limited to, enhancement of the anti-cancer specific immunity at an early stage to kill micrometastases and reduction of the tumor size for easier and safer tumor removal at a later time, especially by surgery.

The first induction radiation is different from traditional radiotherapy, which is intended to kill or minimize cancerous tumors. The goal of the first induction radiation according to the present invention is to trigger the production of neoantigens on the tumor cells in the patient's body (i.e., in situ), thereby triggering the patient's immune system against the tumor cells. A low-dose half-course chemotherapy has a similar purpose. This enhancement of anti-cancer immunity prevents possible metastases caused by the subsequent tumor removal process, e.g., by surgery or biopsy. The dosing amount of the first induction radiation is sufficient to induce neoantigen production on the patient's tumor while avoiding excessive damage to the surrounding normal tissues or interference with surgical wound healing.

The “radiation therapy,” “radiotherapy,” “radiation” or variations thereof as used herein can be internal or external radiation therapy. External beam radiotherapy or teletherapy utilizes an external source of radiation that is administrated through a machine which is capable of producing high energy external beam radiation, such as ionizing radiation. The radiation itself can be, for example, electromagnetic (X-ray or gamma radiation) or particulate (e.g., α or β particles, protons, or neutrons). Internal radiation therapy, or brachytherapy, involves implantation of a radioactive isotope as the source of the radiation. Methods of delivering internal radiation sources, and the choice of such implant are within the purview of those skilled in the art. In some embodiments, intensity modulated radiation therapy (IMRT) is utilized as a first induction radiation according to the present invention. Preferably, the first induction radiation is in the form of precision radiotherapy such as Stereotactic Body Radiation Therapy (SBRT). SBRT is considered a more precise method of targeting tumors than conventional radiotherapies. SBRT can deliver higher and more focused radiation dose on tumors with less damage on surrounding tissues.

In some embodiments, the first induction radiation is delivered in the form of radioactive iodine-131 (¹³¹I) at a preferred dose of about 1.1-5.5 GBq. In some embodiments, the first induction radiation delivers radiation in the total dose of about 8 Gy to about 40 Gy of ionizing radiation depending on the locations and types of solid tumors. A preferred total dose is about half of the total dose of the conventional radiotherapy, the preferred dose ranging from about 24 Gy to about 36 Gy, or from about 24 Gy to about 32 Gy. In other embodiments, SBRT is delivered in the total dose of about 24 Gy to about 36 Gy. These ranges are sufficient to induce an “in situ vaccine” while not affecting the wound healing in case of a later surgery. Generally preferred ranges for human patients are from about 24 Gy to about 36 Gy, or from about 24 Gy to about 32 Gy.

Furthermore, the first induction radiation may be hypofractionated. Inventors have surprisingly discovered that production of neoantigens can be induced effectively at a dose starting at around 4 Gy per fraction, more preferably at 8 Gy per fraction. Preferably, the first induction radiation is hypofractionated into about 4 Gy to about 16 Gy per fraction, preferably about 4 Gy to about 12 Gy per fraction, and more preferably about 4 Gy to about Gy per fraction. Typically, the radiation is provided in the dose rate of about 0.5 Gy/min to about 10 Gy/min, preferably about 4 Gy/min to about 6 Gy/min.

In some embodiments, the first induction radiation is delivered in about 4 Gy to about Gy per fraction. In other embodiments, the first induction radiation is delivered in about 6 Gy to about 12 Gy per fraction. In specific embodiments, the total dose of the first induction radiation is a single radiation of 8 Gy. In other specific embodiments, the total dose of the first induction radiation is 40 Gy which is provided in about 5 to about 8 fractions. Preferably, SBRT is delivered in the total dose of about 24 Gy to about 36 Gy, and is hypofractionated into about 4 Gy to about 12 Gy per fraction.

Generally, after completion of the first induction radiation, the time required for the generation of neoantigens and enhancement of anti-cancer immunity is about 3-8 weeks, preferably, about 4-6 weeks.

In other embodiments, the first step of the treatment method further comprises a targeted therapy, a low-dose, half-course chemotherapy, and/or administration of immunoenhancers, immunomodulators, anti-vascular drugs, and/or immune checkpoint inhibitors (ICI) to further promote the in situ production of tumor neoantigens. Targeted therapy may be desirable, for example, if there is a clearly defined oncogene or oncopathway, to enhance the killing of cancer cells, especially possible micrometastases. Exemplary immunoenhancers include but are not limited to granulocyte-macrophage colony-stimulating factor (GM-CSF), IL-2, thymosin 28 peptide or 5 peptide, and Chinese Medicine. Exemplary immunosuppressive blockers include but are not limited anti PD-1, anti PD-L1, anti CTLA-4, and anti CD47. In specific embodiments, the first induction radiation is combined with a granulocyte macrophage colony-stimulating factor (GM-CSF) to enhance the patient's immune response. Other treatment method may be combined with the first step of the invention, as long as it does not substantially interfere with the goal to induce an “in situ vaccine” without damage to the host immune system.

Second Step

The second step of the treatment method according to the present invention comprises removal of tumor tissues from the patient, through surgery, biopsy, or any other means known in the art. The timing of the tumor tissue removal is about 3 weeks to about 8 weeks, preferably about 4 weeks to about 6 weeks after the completion of the first induction radiation or a low-dose half-course chemotherapy. In some embodiments, a test such as the immunoassay protocol of the present invention is run prior to the tumor removal in order to confirm that the patient has increased their anti-cancer immunity as a result of the first induction radiation or a low-dose half-course chemotherapy.

Removal of the tumor tissue may coincide with only a partial removal of the detectable tumor, complete removal of detectable tumor, or debulking. If a patient has multiple lesions of cancerous tumor that cannot be removed or difficult to remove, a tumor tissue may be biopsied, for example, in the amount of about 10 mg to about 20 mg. Because the first induction radiation according to the present invention works to stimulate the production of new neoantigens on the patient's tumor, i.e., the in situ vaccine, there is a strong likelihood of the induced neoantigens having high immunogenicity, resulting in the activation of the patient's immune response against the tumor. The induced neoantigens contain a large amount of “pro-growth and anti-death” oncoproteins that are useful for the individualized tumor vaccine. Thus, benefits of the combined first and second steps of the cancer treatment method according to the present invention include, but are not limited to: 1) a reduced likelihood to experience metastasis or proliferation of residual cancerous cells that can be otherwise caused by the removal process, because the anti-cancer immunity has been enhanced by first induction radiation; 2) obtainment of the patient's own tumor cells that can be cultured while being induced in vitro in the next step to produce additional newly formed neoantigens that can be used for a self-tumor vaccine.

Third Step

The third step of the treatment method according to the present invention comprises a preparation of a self-tumor vaccine comprising in vitro-induced neoantigens. As described previously and without being bound by a theory, the inventors hypothesized that an existing tumor has already and at least partially escaped the body's “immune surveillance,” and thus it is necessary to induce production of additional neoantigens on the tumor tissue by culturing the tumor tissue in vitro under a “survival pressure.” For this, the removed tumor tissues are prepared into single-cell suspensions using techniques known in the art. A preferred method is mechanical shear or grinding, which does not introduce additional proteins unlike enzymatic methods. The cells are cultured for at least about 24-48 hours, during which the suspended cells are subjected to a “survival pressure.”

“Survival pressure” refers to a condition that induces production of neoantigens on the treated culture cells. In preferred embodiments, the survival pressure also induces heat-shock proteins and other antigen presenting molecules, which enhance the presentation of neoantigens to host immune cells. In some embodiments, the survival pressure is created by a cell irradiator according to the present invention, which is capable of delivering to the cells a second induction radiation in the form of an X-ray radiation and in the total dose ranging from about 6-10 Gy, more preferably about 8 Gy. The radiation is typically done in one sitting. The dose rate may be between about 0.5 Gy/min. and about 5 Gy/min.

Optionally, other devices or means may be used for the second induction radiation to produce X-ray, gamma ray, or other forms of radiation that causes sufficient damage to the DNA to induce mutation and neoantigen production. In other embodiments, the survival pressure is created by other physical means (hypoxia, high temperature, etc. or any combination thereof) as long as they induce expression of more neoantigens by the cultured cells. For example, hypoxia is created in an incubator at 1% oxygen, and high temperature can be about 39-40° C.

The duration of the above treated cell culture is between about 1 and 3 days, depending on the tumor origins, cell types, and degree of cell proliferation. Typically, the duration of cell culture according to the present invention is about 2 days.

The cells are thereafter harvested, inactivated, and homogenized as cell lysate by appropriate methods known in the art. The inactivation of cells may be performed by, for example, ultrasonic cell inactivation method, repeated cell freeze-thaw, low permeability rupture, homogenization, or a combination thereof. The protein content in the cell lysate can be determined by methods known in the art, such as the BCA (Bradford protein assay) method. Advantageously, the method according to the present invention does not involve separation of neoantigens from the cell lysate. The tumor cell lysate is then divided into suitable aliquots in vials. In some embodiments, the total amount of tumor cell lysate comprising the whole set of induced neoantigens is about 1 mg per vial or about 2 mg per vial. Preferably, the vial further comprises about 0.5 mL of about 1 mg to about 2 mg of cell lysate.

The tumor cell lysate in vials can be cryopreserved at about −80° C. until it is ready to be administered to the patient from whom the tumor tissue originated. The total amount of cell lysate that is obtained from the process depends on the size and weight of the removed tumor.

Fourth Step

The fourth step of the treatment method according to the present invention comprises administration of the self-tumor vaccine to the patient from whom the tumor tissue was obtained. It is within the purview of those skilled in the art to determine the timing of the self-tumor vaccine administration, as it is dependent on various factors including the patient's clinical situation and the anti-cancer treatment being provided under the current guideline. Typically, if the tumor is still in its early stage without metastasis, and thus there is no need for a systemic treatment, the self-tumor vaccine may be administered within days or about 1 to 2 weeks after the tumor tissue removal. If tumor residue or metastasis is observed in the patient, the systemic treatments such as conventional chemotherapy and/or targeted drugs may be necessary. In case of conventional chemotherapy, the use of vaccine may be postponed until the white blood cell (WBC) count recovers, which is typically about 1 month to about 2 months after the completion of chemotherapy because the cytotoxic drugs would destroy the patient's WBC count, weakening the immune system. In case of targeted drugs such as tyrosine kinase inhibitors (TKI), growth factor receptor antibodies (such as Trastuzumab or Bevacizumab), or their pathway inhibitors, they have little harm on WBC, therefore, the self-tumor vaccine may be administered sooner or concurrently with the targeted drugs. On the other hand, cellular anti-cancer treatments (e.g., Chimeric antigen receptor (CAR) T-cell therapy) commonly have severe side-effects, and therefore, the self-tumor vaccine may not be administered until such treatment is completed.

For preparation of a self-tumor vaccine for administration, the fresh tumor cell lysate is used or if it was frozen, it is thawed at room temperature immediately prior to administration. In some embodiments, at least one adjuvant is added to the vial after thawing and mixed well. Preferably, the adjuvant is added in an equal volume as the solution of cell lysate, i.e., in 1:1 ratio.

Adjuvant as used herein means an ingredient that can be mixed with the cell lysate in the same vial to make a vaccine or administered to the patient separately from the vaccine to create a stronger immune response in the patient receiving the vaccine. In some embodiments, the adjuvant is selected from liposomes, cytokines (such as about 10 mg to about 100 mg of GM-CSF, CSF, IFNγ, IL-2, IL-9, IL-12, etc.), emulsifiers, or the like. In other embodiments, 100 μg of GM-CSF and/or IL-2 may be mixed with the self-tumor vaccine. In other embodiments, additional 100 μg of GM-CSF and/or IL-2 is separately and subcutaneously injected in the arm of the patient once a week for 4 weeks to enhance the immune response to the self-tumor vaccine. Exemplary adjuvants may also include, but are not limited to, different combinations of various plant proteins, DNA, inactivated viruses, bacteria, fungi, microorganisms, or their metabolites to attract antigen-presenting cells.

The self-tumor vaccine is administered via intradermal injection in all four limbs. In one embodiment, the administered dose is about 0.1 mL of the vaccine per injection site. Each limb is given 1-2 injection sites such that there is a total of 4-8 injection sites in the four limbs. If the patient is missing at least one limb, the number of injection site per remaining limb can be adjusted such that the total number of sites is 4-8. If the patient lacks a limb, it is within the purview of a skilled artisan to make appropriate adjustments.

In some embodiments, the total amount of cell lysate comprising proteins in the self-tumor vaccine per immunization is about 1 mg to about 4 mg. In further embodiments, one immunization comprises about 0.4 mL to about 0.5 mL of about 1 mg to about 2 mg of vaccine proteins mixed with an equal volume of an adjuvant, administered in about 0.1 mL per injection site.

In other embodiments, about 3 weeks to about 4 weeks after the initial “priming” immunization, booster immunizations are provided to the patient in the same manner as the first immunization. Booster immunizations can be regularly given to the patient, about 3-4 times in total, spaced apart at about 3-6 months intervals, until the cryopreserved self-tumor vaccines are used up. If cancer goes into complete remission, the self-tumor vaccine can be intradermally injected about once every 6 months to prevent recurrence.

In some embodiments, the self-tumor vaccine is be administered with immunomodulators such as immunoenhancers (e.g., GM-CSF, thymosin 28 peptide or 5 peptide, Chinese Medicine) and/or immunosuppressive blockers (e.g., anti-PD-1, anti-PD-L1, anti-CTLA-4, anti CD47), and other ICI known in the art. For example, for patients having multiple lesions of cancerous tumors that cannot be removed or are difficult to remove, administration of the self-tumor vaccine according to the present invention can be combined with other treatments such as administration of immunomodulators. The immunoenhancers and/or immunosuppressive blockers can be given to the patient in any combination, and at any timing and schedule as deemed appropriate by the treating physician. Such variations and adjustments are within the purview of those skilled in the art.

If recurrence, oligometastases, and/or new tumor is detected, the above-described four steps of the cancer treatment method can be repeated. Taken together, the treatment method according to the present invention effectively coordinates surgery, chemotherapy, radiotherapy, and immunotherapy in a novel way to create an individualized in situ vaccine as well as a self-tumor vaccine to maximize the anti-cancer immunity in a patient. Since there are heterogenic differences between new and old tumors, repeating the four steps would effectively address any mutations in the neoantigens for treating the new tumor.

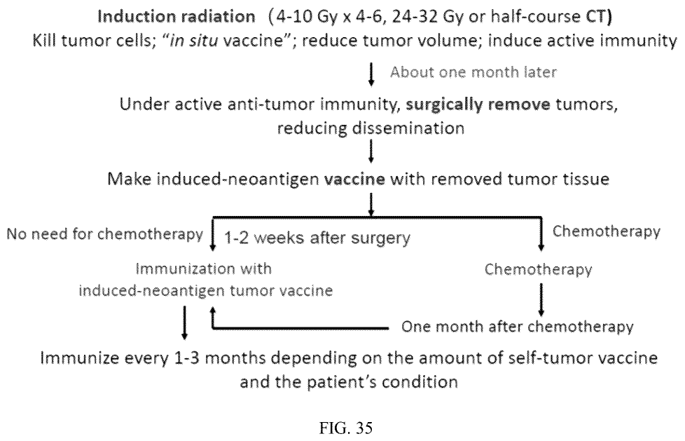

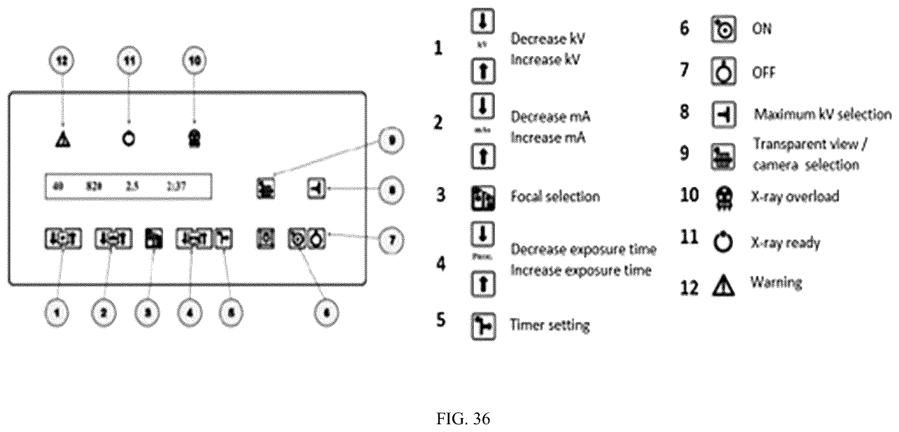

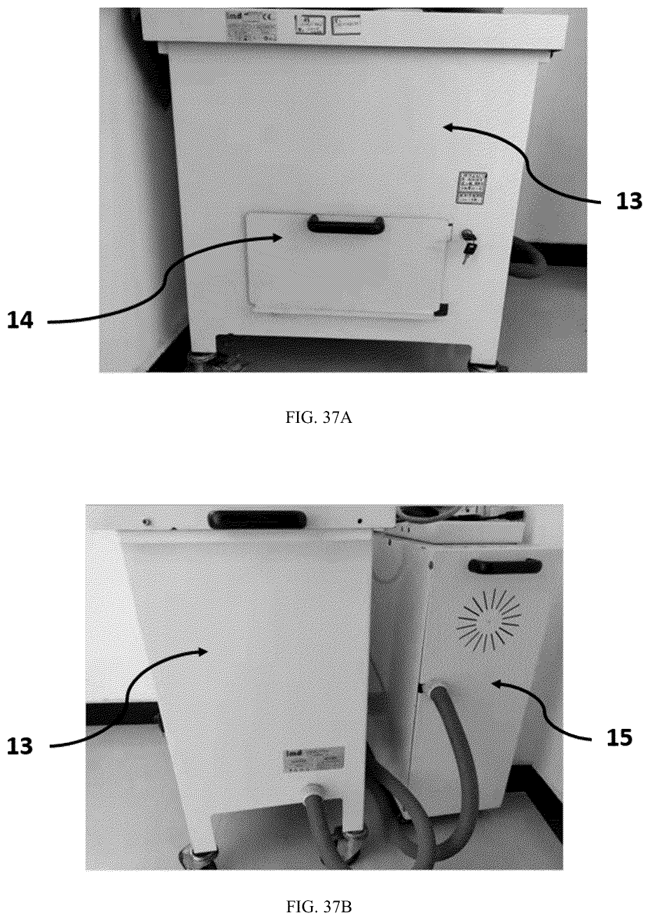

An embodiment of the cancer treatment method according to the present invention is shown in FIG. 35, which is a flow chart explaining the exemplary steps of the method. In the first step, once a patient is diagnosed with cancer, the first induction radiation is applied to the patient, preferably targeting the tumor specifically, to induce production of neoantigens on the tumor, as the “in situ vaccine,” thereby enhancing the patient's anti-cancer immunity and preferably reducing the tumor volume. Alternatively, or additionally, a low-dose half-course chemotherapy can be used with or without GM-CSF and/or immunomodulators. The second step is removal of tumor tissues which occurs about a month after the completion of the first induction radiation. The removal can be done surgically as indicated in the figure, or by biopsy. The enhanced anti-cancer immunity generated by the first induction radiation fights metastases that can be caused by the removal process. The third step of the treatment method according to the present invention comprises a preparation of a self-tumor vaccine comprising neoantigens induced under a “survival pressure” in vitro. The fourth step is administration of the self-tumor vaccine to the patient, the timing of which depends on the patient's clinical situation and the treatment being provided. Generally, however, if there are possible metastases, possible treatment options are radiotherapy, targeted drugs, and/or chemotherapy. If the patient receives cytotoxic chemotherapy, then immunization with the self-tumor vaccine should be postponed at least until about one month after the chemotherapy. For other treatments such as radiotherapy and targeted drugs, immunization with the self-tumor vaccine may commence about 1 to 2 weeks after the tumor removal. In some embodiments, GM-CSF as an adjuvant is mixed with or administered concurrently with the self-tumor vaccine. Depending on the amount of the remaining self-tumor vaccine and the patient's condition, booster immunizations can be given to the patient thereafter about 1 month after the priming immunization. The booster may be repeated about every 1-3 months thereafter. The duration of intervals between boosters may deviate from this general range. In some embodiments, boosters may be given 2 times with 1 week interval for the second booster. In further embodiments, a 6-month interval may be had before the third and later boosters. In some embodiments, changes in the humoral and cellular immunity of the patient are preferably monitored and analyzed with the immunoassay protocol of the present invention, preferably throughout the entire treatment course.

### Immunoassay Protocol for Determining a Patient's Anti-Cancer Immunity

Another aspect of the invention is directed to a set of immunoassay protocols and methods of using same to monitor changes of the humoral and cellular anti-cancer immunity caused by cancer treatments. Another aspect of the invention is directed to an immunoassay kit that incorporates the tests and analyses required by the protocol. The kit enables one to evaluate the anti-cancer immunity of a patient as described herein. Optionally, the kit further comprises a CTC test.

The immunoassay protocol of the present invention evaluates three aspects of the anti-cancer immunity from a patient's blood sample: (1) humoral immunity; (2) cellular immunity; and (3) circulating tumor cells (CTC). Both the humoral immunity and cellular immunity must be evaluated, while CTC is optional. Major components of the immune system in human are lymphocytes which include T cells and B cells. T cells are generally involved in cell-mediated immunity (cellular immunity), whereas B cells are primarily responsible for humoral (antibody-driven) immunity. B cells respond by producing antibodies, whereas T cells (e.g., T helper cells) become activated when they are presented with peptide antigens on the surface of antigen-presenting cells (APCs). Once activated, T cells divide rapidly and secrete cytokines or granules containing enzymes to induce the death of cancer cells (e.g., by cytotoxic T cells). Thus, each component of the immunoassay protocol is designed to measure different immunological parameters of a patient.

Every cancer patient possesses different degrees of immunological parameters that can be detected even if the patient's immune system is not strong enough to eradicate cancer. However, there is currently no systematic method to assess the changes in a patient's immunity against cancer in response to a treatment. Thus, the present invention provides a systematic method of evaluating the appropriate parameters indicative of the patient's immune response to a treatment.

Generally, the more effective a treatment is, the more tumor cells are killed, and the stronger anti-cancer immunity is triggered. Monitoring the anti-cancer immunity in terms of humoral immunity, cellular immunity, and/or CTC before and after treatments serves important purposes: 1) to explain why the same treatment has different outcomes in different patients, which might be due to different immunity responses to a given treatment; 2) to predict the outcome of a treatment or determine whether the tumor is drug sensitive. The more neoantigens released from tumor cell death, the stronger anti-cancer immunity is triggered to suppress cancerous tumor; 3) to re-adjust the treatment approaches if necessary, such as by using more effective tumor-killing drugs or enhancing host immunity by GM-CSF or other drugs. In some embodiments, the immunoassay protocol is useful in understanding the effects of the induced neoantigen vaccines of the present invention on the patient's anti-cancer immunity and determining the next steps if necessary.

The results of the immunoassay protocol are preferably analyzed against the patient's pre-treatment state of immunity to determine the effects of the treatment. Thus, the protocol according to the present invention requires the patient's blood samples obtained, preferably at or around two or more time points discussed above. Preferably, one time point occurs prior to the commencement of a treatment and at least one other time point occurs during or after the treatment. Additional blood samples may be collected at additional time points. In specific embodiments, the patient's immunological parameters are measured from blood samples collected immediately prior to a treatment, and also on a regular (e.g., monthly) basis for up to 3 months after the treatment.

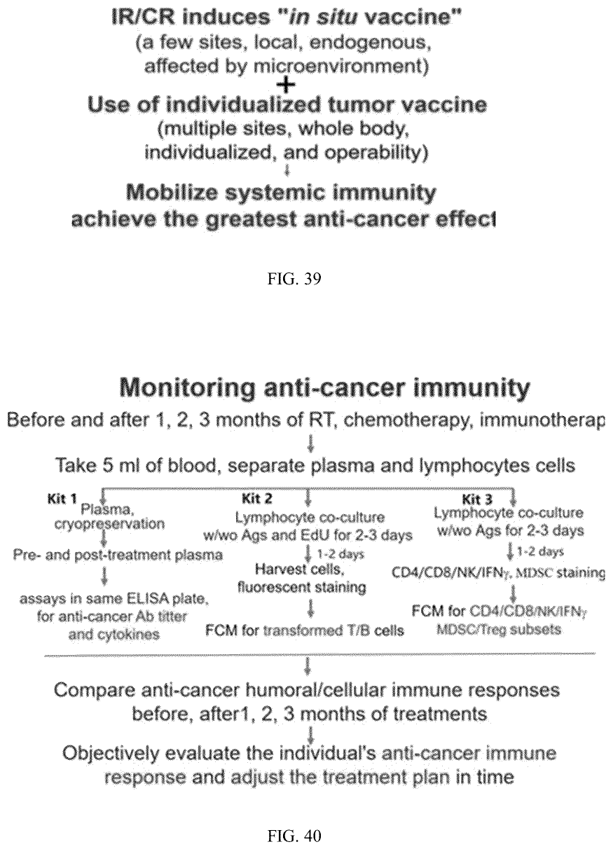

In an exemplary embodiment, about 5 mL to about 10 mL of the patient's blood sample is obtained prior to a treatment. The sample as a whole blood can be analyzed for CTC. Preferably, the sample blood is separated into plasma and lymphocytes (such as peripheral blood mononuclear cells (PBMC) comprising lymphocytes) using conventional techniques known in the art. If necessary, the separated plasma and lymphocytes are cryopreserved at −80° C. until they are ready to be thawed and analyzed. Separated plasma is used to determine the patient's humoral immunity, whereas separated lymphocytes are analyzed to determine the cellular immunity. As for the patient's blood samples obtained after the commencement of the treatment, about 5 mL to about 10 mL of blood is obtained. Again, the sample as a whole blood can be analyzed for CTC. Preferably, the sample blood is separated into plasma and lymphocytes in the same manner as the pre-treatment samples. In some embodiments, post-treatment samples are collected after 3, 5, 8 weeks after the treatment. In other embodiments, post-treatment samples are collected after 1, 2, 3 months after the treatment. As with the pre-treatment samples, separated plasma is used to determine the humoral immunity, whereas separated lymphocytes are analyzed to determine the cellular immunity. FIG. 40 provides an exemplary overview of an embodiment of the immunoassay protocol evaluating humoral immunity and cellular immunity according to the present invention. Additional details regarding the evaluation of humoral and cellular immunity are provided below.

Evaluation of Humoral Immunity

The humoral immunity component of the immunoassay protocol according to the present invention comprises assessments of anti-cancer antibody titer and/or cytokine levels in the plasma of a patient. Preferably, the anti-cancer antibody titer and/or cytokine assessment is performed using plasma samples obtained at least once before and once or more after the commencement of a cancer treatment. A preferred technique to use for the humoral immunity evaluation is Enzyme-Linked Immunosorbent Assay (ELISA). If any samples are being compared (e.g., comparison of pre- and post-treatment samples), they are preferably loaded to the same ELISA plates to minimize inter-assay variations.

In some embodiments of the present invention, antigens used to detect the antibodies in the patient's plasma are the neoantigens from the patient's self-tumor vaccine, or if not available, the same type of tumor cells that have undergone a “survival pressure” (“induced neoantigens”). For example, ELISA wells are coated with such neoantigens before a plasma sample is loaded. The “survival pressure” is as discussed in connection with the treatment method of the present invention above. The tumor cells can be cells that were obtained from the patient whose plasma is to be tested, or cells that are similar or equivalent to the patient's tumor cells. Thus, the anti-cancer antibody titer test according to the present invention can be highly individualized for each patient. If the immunoassay protocol is used in conjunction with the cancer treatment method of the present invention, the tumor cells from which the coating neoantigens are obtained are preferably also used to make a self-tumor vaccine.

In other embodiments, the levels of specific cytokines such as IL2, IFNγ, soluble-CD25, and the like may be observed and quantified using a regular commercial-ELISA as markers for anti-cancer immunity.

Preferably, comparisons of antibody titer and/or cytokine are done between the samples obtained before and after the commencement of a treatment to detect changes in the levels of antibodies and/or cytokines, as indicators of the patient's anti-cancer immunity. In some embodiments, an increase of about 20% or more of the antibodies and/or cytokines after the commencement of a treatment as compared to before the treatment is regarded as enhanced anti-cancer immunity and thus a sign that the treatment is effective. Generally, the higher the percentage of the increase, the stronger the anti-cancer immunity being triggered by the treatment method.

In additional embodiments, ELISA analysis for cytokines and antibodies may be further performed on the supernatant of lymphocytes cultured in the presence or absence of induced neoantigens in connection with the lymphocyte transformation test as discussed below. The cell culture with or without induced neoantigens is as discussed under “Evaluation of cellular immunity” below. The supernatant of cells cultured without induced neoantigens is used as a background to be subtracted from the samples subjected to neoantigen stimulation.

Evaluation of Cellular Immunity

The cellular immunity component of the immunoassay protocol according to the present invention comprises a neoantigen-activated lymphocyte transformation (LT) test and/or a set of neoantigen-activated lymphocyte subset tests involving co-culture, followed by an analytical method such as fluorescent staining and detection with flow cytometer.

LT test according to the present invention provides qualitative and quantitative measurements of transformed T-cells and B-cells as indicators of the cell's response to tumor neoantigens that had been produced in vitro in response to a survival pressure. Without being bound by a theory, the assumption here is that T cells and B cells undergo transformation because they have been previously sensitized by the same neoantigens that had been produced by a treatment, for example, the first induction radiation. Upon re-exposure to the same antigens, the T cells and B cells mount specific and rapid proliferation responses against the source of such antigens. The measurement obtained from the cells cultured without the neoantigens is used as a background in this experiment to be subtracted from the detected cells cultured with the neoantigens.

In order to conduct the LT test according to the present invention, freshly isolated or thawed (if previously cryopreserved) lymphocytes (such as PBMC comprising lymphocytes) are divided into two sets of co-culture wells: those cultured in the presence of neoantigens that were induced on tumor cells by a survival pressure (“induced neoantigens”); and those cultured without such neoantigens. Here, the induced neoantigens serve as mitogens to induce lymphocyte proliferation. The “survival pressure” is as discussed in connection with the treatment method of the present invention above. The tumor cells can be cells obtained from the same patient from whom the lymphocytes are obtained or if not available, equivalent cells can be used. Preferably, the tumor cells are obtained from the patient from whom lymphocytes originated. In some embodiments, the induced neoantigens that are incorporated in a self-tumor vaccine according to the present invention are used for the lymphocyte culture.

The amount of the induced neoantigens added to each well is from about 1 μg/mL to about 20 μg/mL, more preferably from about 4 μg/mL to about 10 μg/mL. In some embodiments, the lymphocytes are cultured for about 2-3 days without bovine serum, more preferably for about 2 days. Proliferation of lymphocyte is then detected, for example with a marker such as 5-Ethynyl-2′-deoxyuridine (EdU) or the like having a fluorescent tail. The cells are monitored with known techniques such as flow cytometry (FCM) to detect the EdU being incorporated into the DNA of proliferating cells. In some embodiments, the number of proliferated cells (e.g., EdU⁺ cells) in the culture without induced neoantigens (“Ag⁻”) may be used as a background to be subtracted from the proliferated cells cultured with the induced neoantigens (“Ag⁺”).

In some embodiments, the LT is performed on the lymphocytes obtained from a patient at least two time points, for example, once before and once after the commencement of a treatment. Where samples from at least two timepoints are collected, the number of EdU⁺ cells in the culture without the neoantigen (“Ag⁻”) is subtracted from the lymphocyte transformation. The percentage increase in lymphocyte transformation between two time points is calculated based on the following formula:

[(Ag⁺−Ag⁻)_{time 2}−(Ag⁺−Ag⁻)_{time 1}]/(Ag⁺−Ag⁻)_{time 1})×100% (I)

wherein “time 1” denotes a timepoint that is earlier than “time 2.” Preferably, time 1 is a timepoint prior to the commencement of a cancer treatment. If the percentage increase of LT is more than about 20%, the patient's anti-cancer immunity is considered to have been triggered. The higher the percentage of increase in the number of EdU⁺ cells in the neoantigen-treated culture, the stronger the anti-cancer immunity being triggered in the patient.

In place of or in addition to LT, evaluation of lymphocyte subsets can be performed to assess the cellular immunity of a patient. In some embodiments, an immunofluorescence analysis is performed to detect in qualities and quantities of the compositions of lymphocyte subsets that are activated in a cell culture with or without the induced neoantigens. The cells cultured without the induced neoantigens is used as a background to be subtracted from the detected cells cultured with the neoantigens.

To analyze lymphocyte subsets, freshly isolated or thawed lymphocytes are divided into two sets of co-culture wells and cultured the same way as the cells used for LT. The amount of the induced neoantigens used is the same as LT. Preferably, the neoantigens that are used to culture one set of the lymphocytes are derived from isolated tumor cells obtained from the same patient from whom the lymphocytes are obtained. In some embodiments, the same neoantigens that are used to make a self-tumor vaccine according to the present invention are used for the lymphocyte culture.

The lymphocytes are cultured for about 2-3 days without bovine serum, more preferably for about 2 days. In other embodiments, the cells in the culture can be stained with fluorescence-labeled antibody or reagents, i.e., FITC-conjugated-anti-CD8, anti-CD4, anti-natural killer (NK) cells, anti-CD25, anti-MDSC to detect specific subsets of immune cells (e.g., CD8⁺IFNγ⁺, CD4⁺IFNγ⁺, CD56⁺IFNγ⁺, MDSC, Treg), and some double stained with PE-anti-IFNγ (as activation indicator), as indicators of the patient's activated anti-cancer cellular immunity. The stained cells are analyzed with techniques such as flow cytometry (FCM) to determine the qualities and quantities of the activated immune cells in each culture.

In some embodiments, the lymphocytes for subset determination are obtained from a patient at two different time points, preferably at least once before and at least once after the commencement of a treatment. The time points can be the same or different from the time points in LT. In some embodiments where samples from at least two timepoints are collected, the amount of particular subset(s) of cells in the culture without the induced neoantigens (“Ag⁻_{cell}”) is subtracted from the amount of cells cultured with the neoantigens (“Ag⁺_{cell}”) to assess the increase in the particular types of cells. The percentage increase in a particular cell type is calculated based on the following formula:

[(Ag⁺_{cell}−Ag⁻_{cell})_{time 2}−(Ag⁺_{cell}−Ag⁻_{cell})_{time 1}]/(Ag⁺_{cell}−Ag⁻_{cell})_{time 1})×100% (II)

wherein “time 1” denotes a timepoint that is earlier than “time 2.” If the percentage increase of at least one cell type is more than about 20%, the patient's anti-cancer immunity is deemed to have been triggered.

Circulating Tumor Cells (CTC)

Optionally as part of the immunoassay protocol of the present invention, circulating tumor cells (CTC) are measured as an indicator of the tumor killing capacity of a patient's induced anti-cancer immunity. Preferably, the blood samples to be analyzed are collected at least once before and at least once after the commencement of a treatment. The CTC test is carried out in a manner known in the art.

The results of the tests to evaluate the humoral immunity, cellular immunity, and/or CTC are analyzed to multifacetedly assess the degree and types of anti-cancer immunity that was developed as a result of a treatment provided to the patient.

Generally, the higher the percentage of activated immune cells and/or the humoral parameters in a patient after the commencement of a treatment as compared to before the treatment, the stronger the anti-cancer immunity that has been generated as a result of the treatment. In such case, it can be concluded that the patient's current treatment is effective. In some embodiments, an increase in a patient's anti-cancer immunity is determined if at least about 20% increase in each of the tested parameter(s) is observed in later measurements compared to earlier measurements.

If the results indicate insufficient anti-cancer immune response after a treatment, the treatment should be adjusted. In some embodiments, the conclusion of insufficient response may be drawn based on less than about 20% increase in the measured parameters in later measurements compared to earlier measurements. If the treatment is the self-tumor vaccine according to the present invention for example, its dosage amounts and/or types of vaccine adjuvants may be adjusted. As non-limiting examples, treatment enhancer such as GM-CSF, thymosin, IFNγ, anti-PD-1 may be used concurrently with the self-tumor vaccine.

The results of the immunoassay protocol may also be useful in deciding whether to utilize additional treatment methods, such as applying a low-dose radiation to stimulate bone marrow or other immune organs of the patient, or administering immune-enhancing drugs. Furthermore, the results would aid in determining clinical prognosis.

### Cell Irradiator

In some embodiments of the invention, a cell irradiator is used to generate and apply a “survival pressure” in the form of a second induction radiation to tumor cells removed in the third step of the treatment method described herein. The cell irradiator irradiates the tumor to induce production of neoantigens to enhance tumor antigenicity. The cell irradiator may have different constructions, but generally comprises a high voltage transformer, X-ray tube, and an X-ray chamber for irradiating the tumor cells with a required dose of X-ray. In some embodiments, the cell irradiator is portable.

A high voltage transformer is capable of increasing an electric energy to a sufficiently high voltage (e.g., about 95-110 kV) for creating the X-rays. In one embodiment, the high voltage transformer is coupled with or incorporates an X-ray tube. The X-ray tube uses a high voltage to accelerate the electrons that collide with a metal target for creating the X-rays. An exemplary metal target includes, but is not limited to, tungsten, rhenium, molybdenum, copper, and/or cobalt.

An X-ray chamber provides a space for accommodating and irradiating tumor cells with the X-rays generated by the high voltage transformer. The X-ray chamber is of suitable shape and size so as to readily accommodate the samples to be irradiated. In one embodiment, the internal dimension and volume of the X-ray chamber is about 30 cm×30 cm×45 cm. In other embodiments, the dimension of the X-ray chamber is about 30 cm×30 cm×30 cm.

Preferably, the cell irradiator further comprises a heat dissipation system to timely dissipate heat generated by the high voltage transformer, such that the working temperature of the X-ray tube is maintained within a certain range, preferably between about 10° C. and about 50° C.

Preferably, the cell irradiator further comprises a safety protection system which ensures that the radiation intensity outside the cell irradiator does not substantially increase and remains lower than the internal environment at all times. This can be achieved, for example, by ensuring a full seal of the X-ray chamber when the cell irradiator is in use to prevent X-ray leakage. For example, all six sides of the cell irradiator can be sealed with a lead plate or a lead door to prevent the release of X-ray to the environment. The safety protection system may further provide a mechanism whereby the X-ray emission tube is activated to emit rays only when X-ray chamber is completely sealed.

Preferably, the cell irradiator further comprises a control system to adjust the voltage and current of the high voltage transformer to control the radiation dose rate of the X-ray tube, as well as to control the radiation time. An embodiment of such control system is provided in FIG. 41. Preferably, the control system can adjust the radiation dose rate to be about 0.5 Gy/min. to about 2 Gy/min. FIG. 38 provides a table of exemplary radiation rates of an embodiment of the cell irradiator according to the present invention. Means to control the radiation time may comprise a manual switch and/or a timer that automatically shuts off the radiation after a predetermined time.

In some embodiments, the cell irradiator is capable of delivering X-ray radiation to the cells in the total dose ranging from about 6 Gy to about 10 Gy, preferably from about 6 Gy to about 8 Gy either in one sitting or in fractions. Preferably, the cell irradiator is capable of delivering from about 6 Gy to about 8 Gy of X-ray per fraction to a cell sample.

Preferably, the cell irradiator further comprises a means to focus the generated X-ray on the target sample, a camera to view the inside of the X-ray chamber once it has been closed, and other features that alert the user when the predetermined dose is ready to perform, and/or in case of X-ray leakage or overexposure.

An embodiment of the cell irradiator according to the present invention is shown in FIGS. 41, and 42A-42D. The cell irradiator 13 comprises a high voltage transformer 15 operatively connected to, X-ray tube, and an X-ray chamber 14 with a door for accommodating and irradiating the tumor cells with a required dose of X-ray. The high voltage transformer is capable of increasing an electric energy to a sufficiently high voltage (e.g., about 95-110 kV) for creating the X-rays. The cell irradiator 13 further comprises a heat dissipation system 17 in the form of radiators disposed on top of the transformer 15 to timely dissipate heat generated by the high voltage transformer. The cell irradiator 13 is capable of achieving a full seal of the X-ray chamber 14 when it is in use to prevent X-ray leakage.

The cell irradiator 13 further comprises a control system 16 comprising a means to adjust the voltage 1 and the current 2 of the high voltage transformer 15, as well as the radiation time 4. By adjusting these parameters, the user of the cell irradiator 13 is able to achieve any dose rates as summarized in FIG. 38. The cell irradiator has alternative on/off switch, i.e., the switch without an auto lock 18 for manual operation or the switch with an auto lock 19 that shuts off the transformer 15 after a period of non-use. The control system 16 further comprises a means to focus the generated X-ray on the target sample 3, a means to control a camera to view the inside of the X-ray chamber 9. Additional features are signs to alert the user of X-ray leakage or overexposure 10, when the predetermined dose is ready to perform 11, when the cell irradiator is on or off (6, 7). The Timer setting 5 automatically terminates irradiation after a predetermined time.

### EXAMPLES

Following are Examples which are offered by way of illustration and are not intended to limit the invention. Various modifications or changes in light thereof will be suggested to persons skilled in the art and are to be included within the spirit and purview of this application. Unless otherwise stated, the experimental method without specific conditions is carried out according to methods known in the art or recommended by the manufacturer.

### Example 1—Induction Radiation Induces Tumor Cells to Produce New Neoantigens

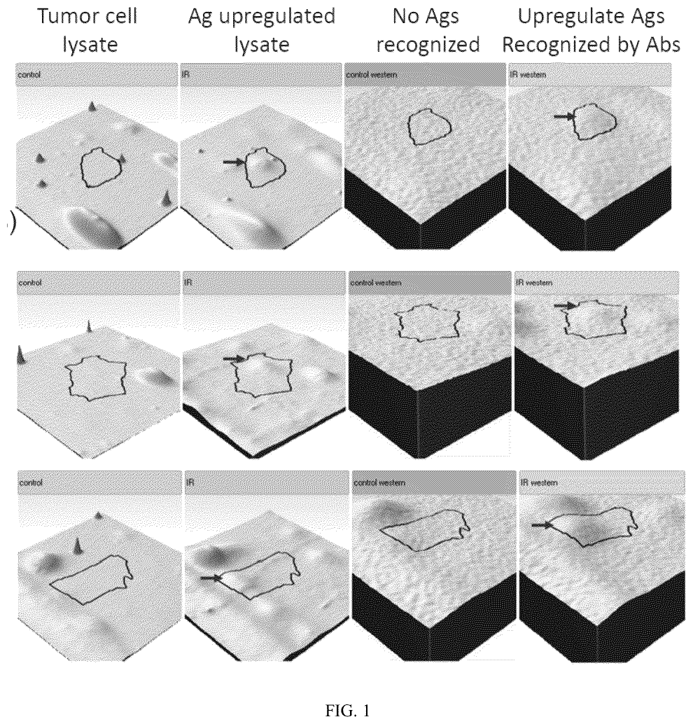

4T1 breast cancer cells were cultured in DMEM (Dulbecco's Modified Eagle Medium) with 10% bovine serum in 5% CO₂ incubator at 37° C. and divided into two groups: one group was treated with 8 Gy induction radiation once (“test group”), and the other group did not receive an induction radiation (“control group”). Two days later, the cells were collected, repeatedly frozen at −80° C. then thawed, and then homogenized by ultrasound to obtain cell lysate comprising antigen proteins under aseptic conditions. Two-dimensional electrophoresis was separately performed on 500 mg of cell lysate from each group, followed the manufacturer's protocols (Bio-Rad) for silver staining, photoimaging, transferring to PVDF membrane. Western blot analysis was performed by staining the PVDF membrane with mouse polyclonal antibodies generated with 8 Gy-irradiated 4T1 cell lysate as an antigen. As shown in FIG. 1, it was found that under the “survival pressure” (i.e., the second induction radiation of 8 Gy), neoantigens were upregulated in the 4T1 breast cancer cells (column 2). In comparison, the control (column 1) showed no sign of increase in the antigen production. Similarly, the Western blot analysis revealed that the group subjected to the survival pressure showed increased neoantigens (column 4) whereas the control group did not (column 3). The results demonstrate that one-time 8 Gy radiation can induce tumor cells to begin producing neoantigens. These neoantigens can induce immune response in vivo.

### Example 2—the Exosome and Lysate of Hep G2 Cells and the Exosomes of the Plasma of Hepatoma Patients Obtained after 8 Gy Induction Radiation Contain New Neoantigens (1)

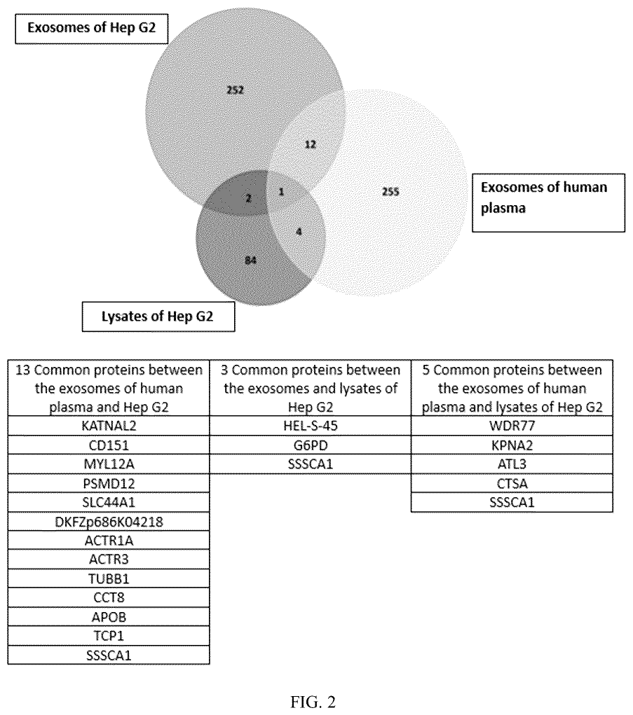

A proteomic study was conducted to assess the types neoantigen expressions in the lysate and exosomes of Hep G2 (a human liver cancer cell line) compared to the plasma of hepatoma patients after an induction radiation. Briefly, the Hep G2 cells were irradiated with 8 Gy once and cultured in a serum-free DMEM. A control group of Hep G2 was cultured in the same manner without being irradiated. Hepatoma patients were treated with 8 Gy SBRT once. Twelve hours after irradiation, the plasma of the patients as well as Hep G2 cells were collected, frozen at −80° C., and shipped for analysis at Shanghai Kangcheng Biotech company. The samples were separated and evaluated with iTRAQ-based quantitative proteomic analysis. The proteins found in the exosome and cell lysate of the irradiated samples of Hep G2 but not in non-irradiated samples of Hep G2 were regarded as neoantigens. As shown in FIG. 2, the proteomic analysis demonstrated that after a single radiation of 8 Gy, the exosomes in the plasma of hepatoma patients shared 17 neoantigens with the exosomes and lysate of the irradiated Hep G2 cells, demonstrating that the induction radiation both in vivo in the patients and in vitro in the cultured cells is capable of triggering the neoantigen production that can be utilized as tumor vaccines.

### Example 3—the Expression of Neoantigens Produced after Induction Radiation is Higher in Tumors than in Adjacent Normal Tissues (2)

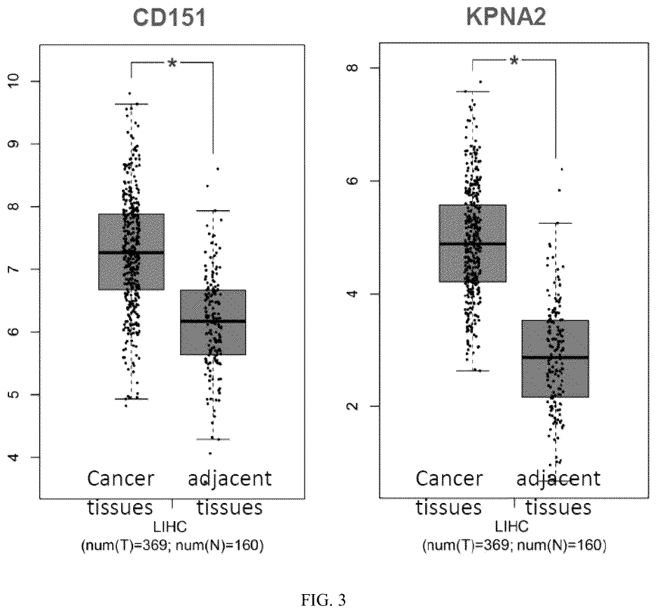

To determine if the radiation-induced proteins, such as CD151 and KPNA2, identified by the proteomic analysis of Example 2, play a role in cancer progression, a comparison of expression levels of these two neoantigens was made between the primary hepatoma tissue and its adjacent normal tissues using the NIH's real-world databases in The Cancer Genome Atlas (TCGA). As shown in FIG. 3, it was found that the expression of radiation-induced neoantigens CD151 and KPNA2 is higher in hepatoma tissues than in its adjacent normal tissues, suggesting that these neoantigens may play a role in promoting cancer and can be utilized as neoantigens for activation of anti-cancer immunity as means for cancer treatment.

### Example 4—the Upregulation of Neoantigens is Related to Poor Clinical Outcomes (3)

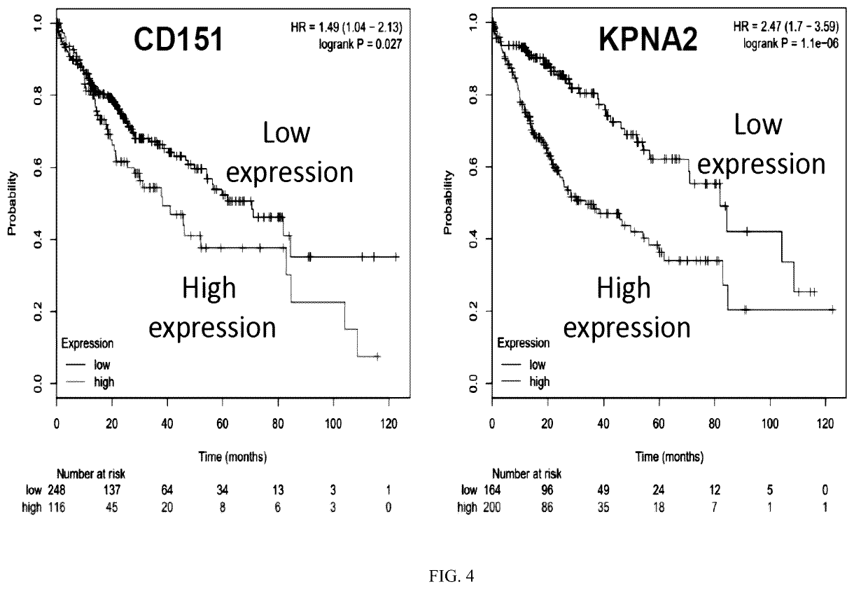

To determine if upregulated neoantigens CD151 and KPNA2 are correlated with poor clinical outcome, a comparison of expression levels of these two molecules was made using the NIH's real-world databases in TCGA. In FIG. 4, the “probability” in the Y axis is the survival rate of hepatocellular carcinoma (HCC) patients and the time in the X axis is the observation time in months. The number of patients with low or high expression level of CD151 or KPNA2 is provided below each graph. It was found that the increased expression of CD151 or KPNA2 was negatively correlated with the outcome of HCC, i.e., the patients with high expression of CD151 or KPNA2 had poor prognosis.

The data in FIG. 2, FIG. 3, and FIG. 4 taken together suggests that, even one dose of 8 Gy of radiotherapy on tumor cells is enough to induce neoantigens that are correlated with tumor progression and poor outcome. Therefore, it is believed that, applying a low total dose of the first induction radiation on the tumors upon cancer diagnosis to produce an “in situ vaccine” can be beneficial for enhancing the patient's anti-cancer immunity upon diagnosis and before the tumor is removed, because the enhanced immunity at early stages of cancer can better prevent local and/or systemic spreading of the primary tumor cells during a tumor removal process.

### Example 5—Experimental Scheme for the Demonstration of the Effectiveness of Radiation-Induced “In Situ Vaccine” Against a Second Tumor Growth (1)

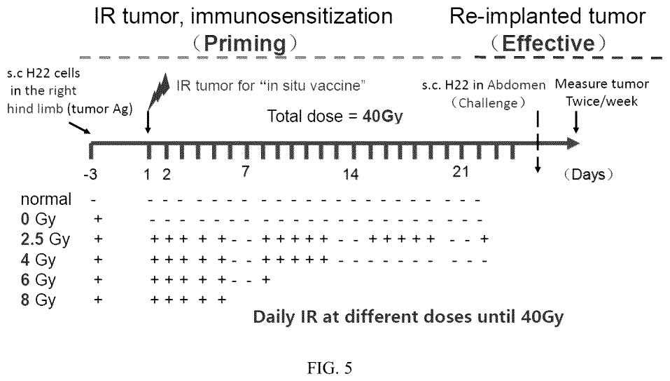

As shown in FIG. 5, an experiment was conducted to assess the immune effects of various doses of induction radiation on second implanted tumors in mice. ICR mice were subcutaneously inoculated with about 1×10⁶ live H22 hepatoma cells in the right hind limb. A different group of ICR mice did not receive tumor inoculation (“normal” group). After 3-6 days, tumors were formed in the hind limb (“first implanted tumor”) in the inoculated group. The inoculated mice were confined to a special plastic container with the hind limb stretched out, and divided into 5 groups. Each group of mice received one of the radiation doses of 0, 2.5, 4, 6, and 8 Gy per day on the tumors (0 Gy indicates no radiation). The doses mimicked different doses used in clinical radiotherapy with an electron accelerator. As is commonly done in clinical settings, each group of mice was irradiated every day for 5 consecutive days followed by two days of no radiation (FIG. 5). For each group, this radiation schedule was followed until the cumulative dose reached 40 Gy, which is about two-thirds (⅔) of a total “cure dose” of radiotherapy. 24 days after the completion of the radiotherapy, each group was subcutaneously inoculated with the same amount of live H22 hepatoma cells in the abdomen as the first inoculation to generate a second implanted tumor. The volume of the second implanted tumor in the abdomen was measured twice a week to determine the effects of the “in situ vaccine” on the growth of the second implanted tumor.

### Example 6—Experimental Scheme for the Demonstration of the Effectiveness of a Radiation-Induced “In Situ Vaccine” Against Metastasis (1)

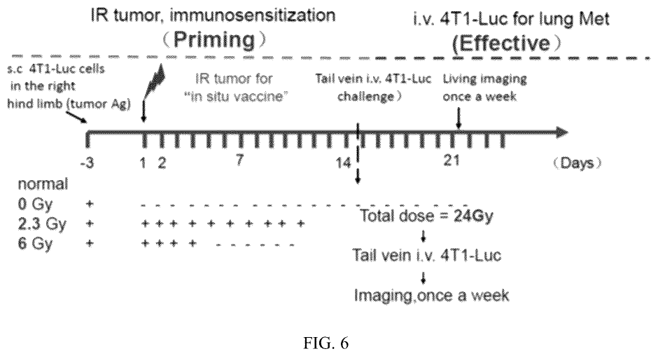

As shown in FIG. 6, an experiment was conducted to assess the effects of an induction radiation on metastasis. The BLAB/c mice were inoculated in the right hind limb with 4T1 breast cancer cells transfected with luciferase as a trace marker for tumor cells. After tumors were formed in the hind limb on day 4, the mice were divided into three groups, each group receiving one of 0, 2.3, or 6 Gy of induction radiation daily on the tumors until the total dose reached 24 Gy, which is one-third (⅓) of a total “cure dose” of radiotherapy. After 3 weeks, each group was injected with 3×10⁵ of 4T1-luciferase cells in the tail vein for formation of lung metastases. After 2-3 weeks, luciferin substrate was injected into the abdominal cavity of each mouse, and the living 4T1-luciferase tumor was observed by in vivo imaging with the IVIS Spectrum. Survival of the mice was also monitored for up to 80 days.

### Example 7—Demonstration of the Effectiveness of Radiation-Induced “In Situ Vaccine” Against Metastasis (2)

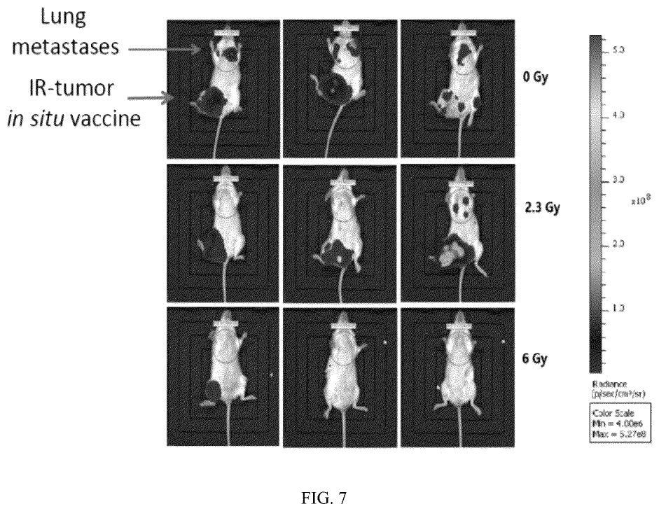

FIG. 7 shows results of Example 6. The group of mice that received 6 Gy/day of induction radiation for a total dose of 24 Gy induction radiation (third row) demonstrated the least number of cells in lung metastases and the fluorescence intensity was the lowest compared to other groups (P<0.05). On the other hand, the mice that received no radiotherapy (first row) demonstrated the least anti-cancer immunity as they had the greatest tumor growth.

### Example 8—Demonstration of the Effectiveness of Radiation-Induced “In Situ Vaccine” Against Metastasis (3)

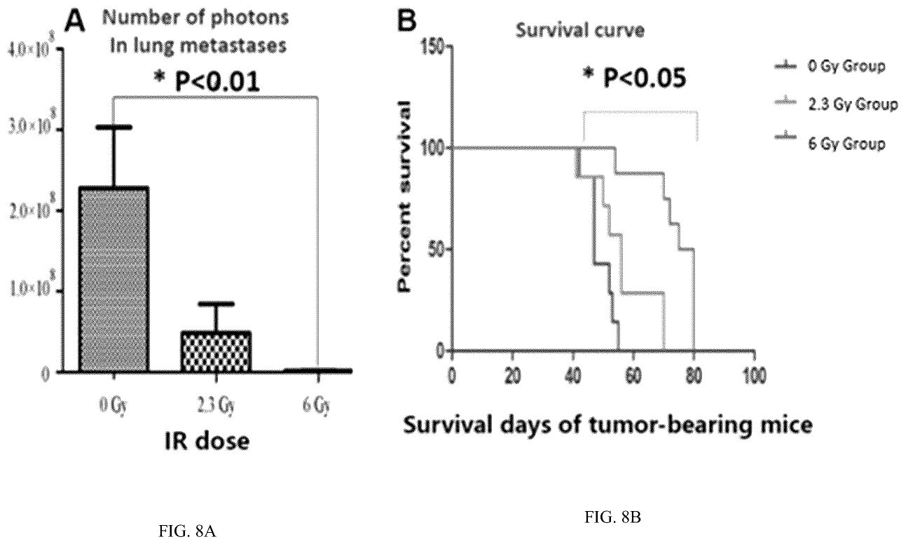

FIGS. 8A-8B show additional results of Example 6. FIGS. 8A-8B further demonstrate the strongest anti-cancer immunity developed by the mice that received 6 Gy of radiation daily for a total dose of 24 Gy induction radiation, as they exhibit the lowest fluorescence intensity (P<0.01) and the longest survival rate (P<0.05). The mice that received 2.3 Gy daily dose of radiation demonstrated some immunity as they had less metastatic cancer overall and less fluorescence intensity (about one-fourth (¼)) compared to the data of the mice that received no induction radiation (P<0.05). The data indicate that an induction radiation with one-third (⅓) of the conventional treatment dose, while it may not be sufficient to cure cancer, can trigger the host's anti-cancer immunity to impede the growth of a second tumor.

Taken together, the results of Examples 5-8 suggest that, applying an induction radiation from about one-third (⅓) to about two-thirds (⅔) of a total “cure dose” of radiotherapy on the primary tumor to generate active anti-cancer immunity before its removal would prevent the residual tumor cells from growing and/or metastasizing.

### Example 9—Increased Anti-Cancer Antibodies and Lymphocyte Transformation Produced by Radiation-Induced “In Situ Vaccine” (4)

To evaluate the anti-cancer immunity induced by the in situ vaccine of Example 6, antibody titer evaluation and lymphocyte transformation (LT) assay were performed.

For antibody titer, a high-binding ELISA plate was coated with 100 μL of 4 μg/mL of 8 Gy-irradiated 4T1 cell lysate, after blocking with 5% BSA-PBS, 100 mL of 1:40000 diluted mouse plasma from each mouse from the 0, 2.3 and 6 Gy groups of Example 6 was added to the antigen-coated wells in triplicates and incubated overnight at 4° C. On the next day, the wells were washed 3 times with 300 mL/well of PBS-0.1% Tween 20, and added with 100 mL of 1:1000 diluted HRP-anti-mouse IgG for 1 hr, and then washed 4 times, followed by 3,3′, 5,5′-Tetramethylbenzidine (TMB) substrate for horseradish peroxidase (HRP) for color development, stopped with 2 M H₂SO₄, and read at A₄₅₀.

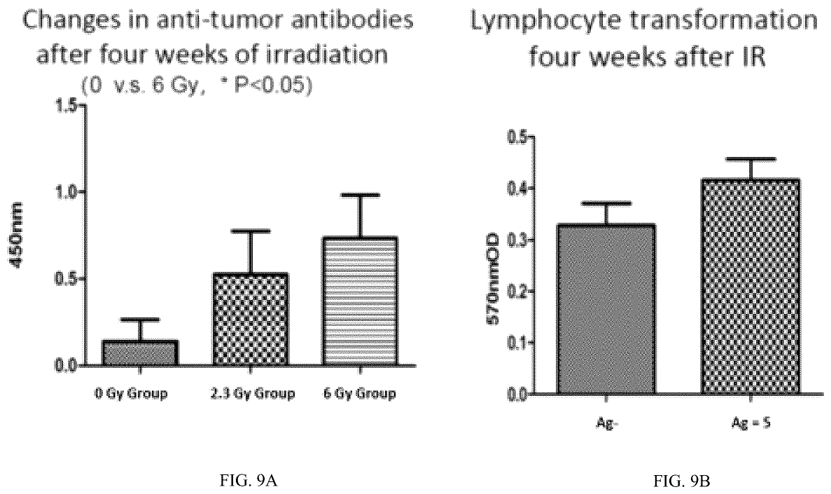

As shown in FIG. 9A, the amount of anti-cancer antibody after four weeks counting from the first day of the first induction radiation was the largest for the group that received a daily dose of 6 Gy induction radiation, followed by the group that received a daily dose of 2.3 Gy induction radiation. The group that received no radiation exhibited the least level of antibodies.

For lymphocyte transformation assay, blood samples were obtained from the mice that received 6 Gy/day in Example 6, 14 days after the induction radiation on the right leg primary tumor. Lymphocytes were separated from the blood using Ficoll and 5×10⁴/well of these lymphocytes were seeded in 24-well plate with 200 μL/well RPMI 1640 media without bovine serum, and then added with or without 20 mL of 400 mg/mL of 8 Gy-irradiated 4T1 cell lysate as mitogen. 2-3 days later, 20 mL of 10 mM MTT was added to each well, cultured for 4 hours, and the transformed blue purple Formazan in proliferated live cells were dissolved in 100 mL DMSO and read at A₅₇₀. As seen in FIG. 9B, the lymphocytes could be activated by the presence of tumor antigens, and the rate of lymphocyte transformation (LT) was increased (P<0.05).

The data presented in FIG. 9A and FIG. 9B indicate that the induction radiation on a primary tumor is capable of inducing the formation of an “in situ vaccine” that triggers the anti-cancer immunity that works to block the formation of a new tumor or metastasis.

### Example 10—Exemplary Embodiment of the Immunoassay Protocol to Assess the Cellular and Humoral Immunity of a Patient Before and After a Cancer Treatment

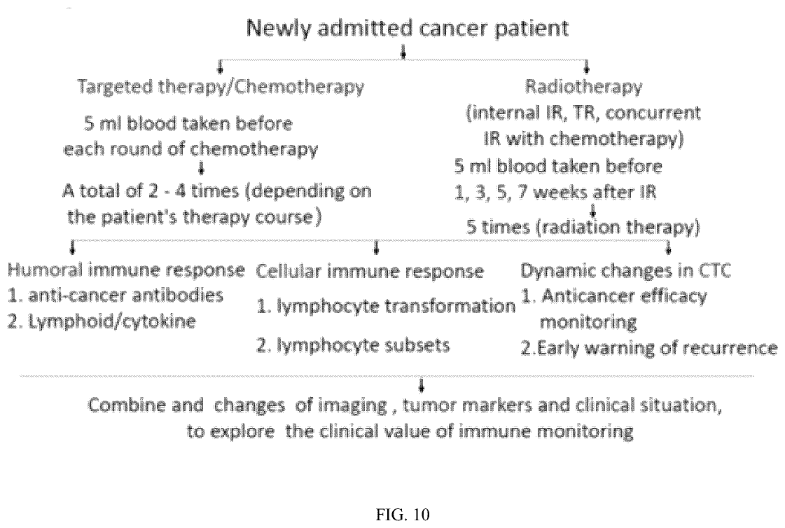

An embodiment of the immunoassay protocol according to the present invention is described in FIG. 10. The protocol can be used to monitor the changes in a patient's anti-cancer immunity induced by a treatment method such as a routine chemotherapy, targeted therapy (such as antibody against growth factor receptors, small molecules blocking signal pathway of tumor promotion, or immune regulation modulators), or radiotherapy, due to its ability to generate an “in situ vaccine” effect. A set of assays is designed to measure the humoral immune response and/or cellular immune response to a given treatment by analyzing the patient's blood samples. The humoral immune response is determined by anti-tumor antibody titer and/or cytokine assay with ELISA, while the cellular immune response can be assessed by the determination of lymphocyte transformation and/or activated lymphocyte subsets such as CD8⁺IFNγ⁺, CD4⁺IFNγ⁺, CD56⁺IFNγ⁺, MDSC, Treg, etc. If desirable, the circulation tumor cells (CTC) is measured as an indicator of the tumor-killing capacity of the patient's induced anti-cancer immunity.

### Example 11—Demonstration of the Immunoassay Protocol (1): Two-Thirds of Patients with Thyroid Cancer had an Increased Anti-Cancer Lymphocyte Transformation (LT) after ¹³¹I Internal Radiation

An immunoassay protocol of the present invention such as the embodiment shown in FIG. 10 was performed on 21 patients who had been diagnosed with thyroid cancer and whose tumors had been surgically removed. Clinically, to prevent metastasis of thyroid cancer, the patients received an intravenous injection of radioactive iodine-131 (¹³¹I) at a dose of 1.1-5.5 GBq once. Immediately after the intravenous injection, the whole-body CT scan was conducted to define the size (big/visible or small/invisible) of possible metastatic thyroid cancer. Blood samples before and 1-2 month after the ¹³¹I internal radiotherapy were collected to evaluate the changes in the patients' anti-cancer immunity.

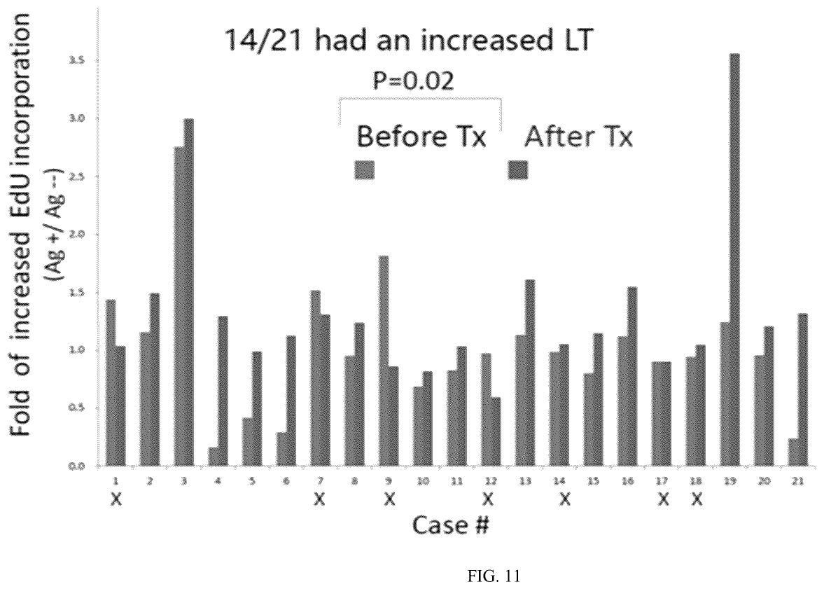

To detect changes in the cellular immunity triggered by the ¹³¹I internal radiation, lymphocyte transformation was assessed according to the immunoassay protocol of the present invention. The lymphocytes obtained before and 1-2 months after the ¹³¹I treatment from each patient were divided into two groups and cultured with or without the 8 Gy-induced neoantigens generated from the patient's removed tumor. As shown in FIG. 11, two thirds (14 out of 21) of the patients not marked with “X” in the figure exhibited an increased lymphocyte transformation as indicated by the elevated incorporation of EdU.

### Example 12—Demonstration of the Immunoassay Protocol (2): The Titer of Anti-Cancer Antibodies Induced by ¹³¹I Internal Radiation was Greater in Patients with a Focal Cancer than Patients with No Visible Lesions

According to the present invention, an antibody titer was measured in the blood samples of the patients of Example 11 before and after the ¹³¹I internal radiotherapy.

The ELISA plates were coated with 100 mL of 4 mg/mL irradiated thyroid cancer cell lysate as antigens. The plasma obtained before and 1 month after the ¹³¹I internal radiation was diluted with PBS at 1:80000, 160000, 320000, 640000, then 100 mL/well of such diluted samples were added to the ELISA plates. After overnight incubation at 4° C., the wells were washed 3 times with 300 mL/well of PBS-0.1% Tween 20, and added with 100 mL of 1:1000 diluted HRP-anti-human IgG for 1 hr, and then washed 4 times, followed by 100 mL of TMB substrate for HRP for color development, stopped with 2 M H₂SO₄, and read at A₄₅₀.

The difference in the antibody titer before and after the ¹³¹I treatment in each patient's sample was divided by his antibody titer before the ¹³¹I treatment measured in the same ELISA plate to determine the percent increase in optical density (OD₄₅₀).

$$\% Increase in O {D}_{450} = \frac{( {OD}_{450} after treatment - {OD}_{450} before treatment )}{{OD}_{450} before treatment} \times 100 \%$$

If % increase in OD₄₅₀ value is greater than 20%, an antibody titer was regarded to have increased.

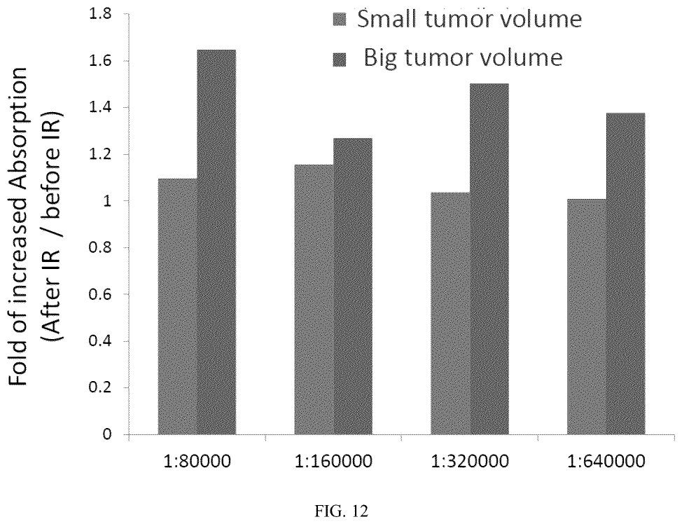

As shown in FIG. 12, the results showed an increased antibody titer was observed after the ¹³¹I internal radiation in patients with visible cancer lesions (indicated as “Big tumor volume”) was larger than that in patients without visible lesions (indicated as “Small tumor volume”). The results suggest that the larger thyroid cancer was, the more cancer cells were killed by the ¹³¹I treatment and the more neoantigens were released, inducing stronger anti-cancer immunity.

### Example 13—Demonstration of the Immunoassay Protocol (3): Increased Levels of Cytokines IL-1β and CXCL16 were Observed in Patients Who Received ¹³¹I Internal Radiation

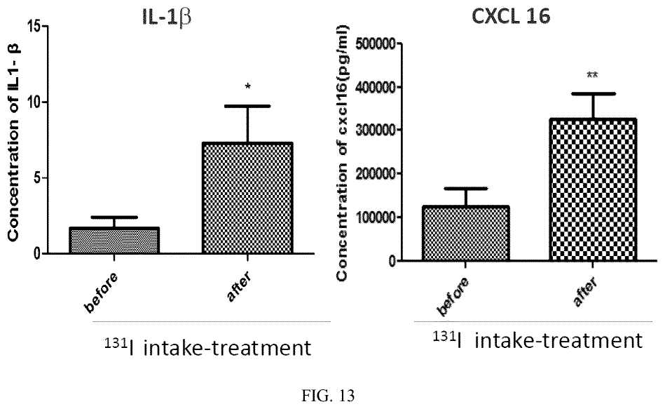

According to the immunoassay protocol of the present invention, changes in the concentration of two cytokines IL-1β and CXCL16 were also measured in the blood samples of the patients of Example 11. The measurements were done by ELISA using the assay kits and protocols from R&D system, Inc. (Minneapolis, USA). As shown in FIG. 13, the levels of IL-1B and CXCL16 increased after the ¹³¹I internal radiation (P<0.05).

Taken together, the data presented in FIG. 12 through FIG. 13 suggest the ¹³¹I internal radiation can induce an anti-cancer immune response in a clinical setting, and the immunoassay protocol according to the present invention is capable of assessing the success of the treatment by comparing the changes in the specific indicators of the anti-cancer immunity before and after the treatment.

### Example 14—the Effects of 8 Gy SBRT Induction Radiation on Hepatocellular Carcinoma (1)—CT/MRI Scan

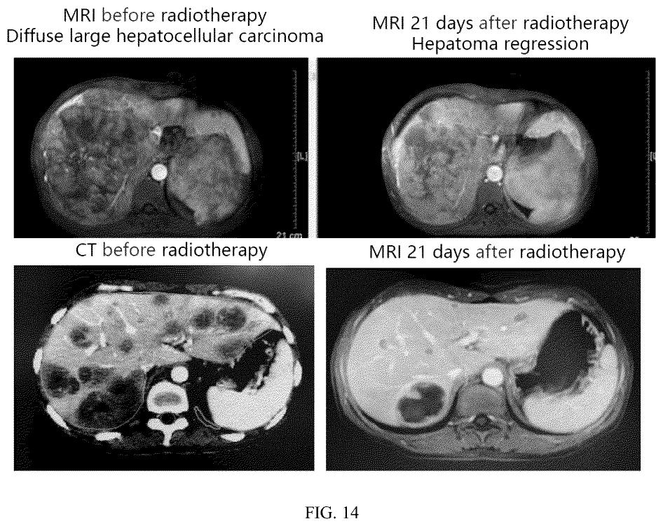

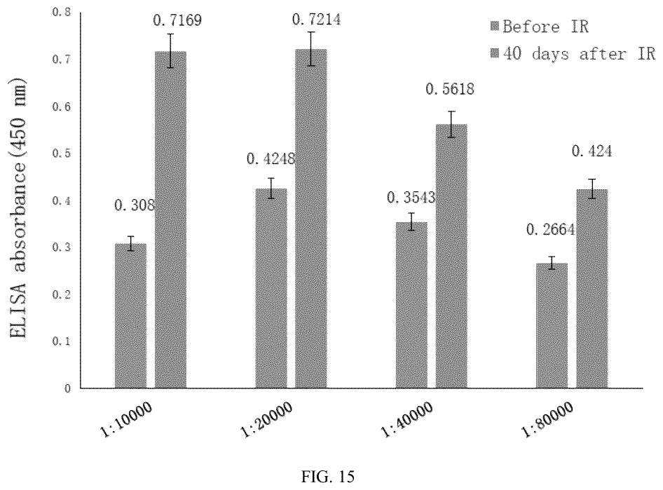

To monitor the anti-hepatoma immunity triggered by 8 Gy SBRT-induced “in situ vaccine,” CT or MRI scan was conducted on a hepatoma patient before and 21 days after a single 8 Gy SBRT treatment. The results in FIG. 14 demonstrate that signs of hepatoma significantly diminished after 21 days of the 8 Gy SBRT induction radiation.

### Example 15—the Effects of 8 Gy SBRT Induction Radiation on Hepatocellular Carcinoma (2)—Lymphocyte Transformation Occurred after 10-43 Days of Radiation

The immunoassay protocol according to the present invention was also performed on the blood samples of the hepatoma patient of Example 14. The blood samples were collected before and after the 8 Gy SBRT.

To assess the changes in the patient's lymphocyte transformation (LT), samples collected from the patient before and after the 8 Gy SBRT treatment were evaluated as an indicator of anti-cancer cellular immunity. The after-treatment blood samples were collected after 1, 10, and 43 days of the single dose, 8 Gy SBRT treatment. For the samples at each time point, 3×10⁵/well of lymphocytes that had been separated from blood samples using Ficoll were seeded in 24 well plate with 1 mL/well RPMI 1640 media without bovine serum, and then cultured with (“Ag⁺”) or without (“Ag⁻” as background) 50 μL of 2 mg/mL of 8 Gy-irradiated hepatoma cell lysate as a mitogen. At the same time, 5 μM of EdU was added to the culture media. After 3 days, the cells were harvested and the incorporated EdU in the live proliferating lymphocytes was detected with staining of EdU according to the manufacturer's instructions, then assayed with C6 flow cytometer (BD Inc., USA). The increased EdU % was calculated according to the following formula:

Increased EdU %=(Ag⁺−Ag⁻)/Ag⁻)×100% (II)

Wherein Ag⁺ and Ag⁻ each represent a count of live lymphocytes incorporating EdU. If the increased EdU % is more than 20%, LT is deemed to have increased.

### Example 16—the Effects of 8 Gy SBRT Induction Radiation on Hepatocellular Carcinoma (3)—the Lymphocyte Subset CD8⁺IFNγ⁺ T Cells Increased Significantly after 43 Days of Radiation

Next, the immunoassay protocol according to the present invention was utilized to assess the changes in the amount of activated CD8⁺IFNγ⁺ T cells in the blood of the hepatoma patient of Example 14, collected before and after the 8 Gy SBRT treatment. The patient's blood was collected before and 1, 10, and 43 days after the 8 Gy SBRT treatment. At each time point, 3×10⁵/well of lymphocytes separated with Ficoll were seeded in 24 well plate with 1 mL/well RPMI 1640 media without bovine serum, and then cultured for 2 days with (“Ag⁺”) or without (“Ag⁻” as background) 50 μL of 2 mg/mL of 8 Gy-irradiated hepatoma cell lysate as a mitogen. Six hours before the cell harvest, 1 μg/mL of GolgiPlug was added to each well to block the secretion of cytokines. Then, the cells were harvested, stained with FITC-conjugated CD8 mAb at 4° C. for 1 hour, washed 3 times with 0.2% albumin-PBS, fixed in 4% paraformaldehyde for 10 min, washed 3 times, treated with 0.1% saponin for 5 min, stained with PE-conjugated anti-human IFNγ⁺ mAb at 4° C. for 1 hour and then analyzed for percentage of double-stained CD8⁺IFNγ⁺ T cells.

### Example 17—the Effects of 8 Gy SBRT Induction Radiation on Hepatocellular Carcinoma (4)—Cytokine Levels in Blood Increased at Different Time Points after Radiation

The immunoassay protocol according to the present invention was utilized to evaluate the changes in the levels of cytokines DcR3, sTNFR, and IL-2 in the plasma collected from the patient of Example 14 before and after 1 and 10 days of 8 Gy SBRT treatment.

### Example 18—the Effects of 8 Gy SBRT Induction Radiation on Hepatocellular Carcinoma (5)—the Titer of Anti-Hepatoma Antibody Remained High 40 Days after the Radiation

The immunoassay protocol of the present invention was further utilized to evaluate the anti-hepatoma antibody titer by ELISA. Briefly, a high-binding ELISA plate was coated with 100 μL/well of 4 μg/mL of 8 Gy-irradiated hepatoma cell lysate containing neoantigens. The plasma samples collected before and 40 days after one dose of 8 Gy SBRT irradiation on the patient's liver were diluted with PBS in 1:10000, 1:20000, 1:40000, and 1:80000, and 100 μL of each such diluted solution was added to the wells in triplicates. After incubated overnight at 4° C., the wells were washed 3 times with 300 μL/well of PBS-0.1% Tween 20, and added with 100 μL of 1:1000 diluted HRP-anti-human IgG for 1 hour, and then washed 4 times, followed by 100 μL of TMB substrate for HRP for color development, then stopped with 2 M H₂SO₄, and read at A₄₅₀.

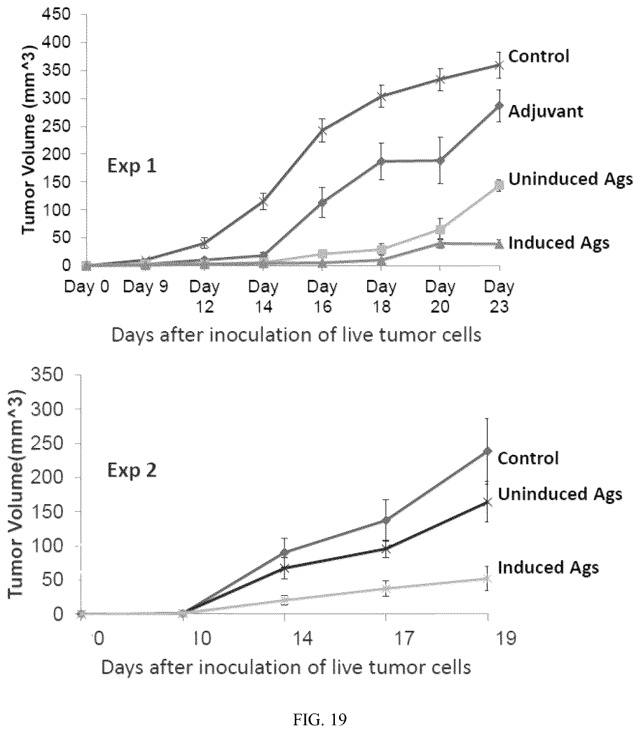

The readings at A₄₅₀ in the same ELISA plate for each dilution were compared before and 40 days after the 8 Gy SBRT treatment. As shown in FIG. 19, the titers of anti-hepatoma antibodies at all different dilutions were higher for the plasma collected 40 days after 8Gy SBRT (“40 days after IR”) than those before the radiation (“Before IR”).

The data of Examples 15-18 demonstrate that even a single 8 Gy radiotherapy is enough to induce an “in situ vaccine” to trigger an active anti-cancer immunity, which can be monitored by evaluating the cellular and humoral immunities according to the immunoassay protocol of the present invention. The conclusion is consistent with CT and MRI of the patients taken before and 21 days after the 8 Gy SBRT treatment as shown in FIG. 14 (Example 14).

### Example 19—Evaluation of Dynamic Changes in the Anti-NPC (Nasopharyngeal Cancer) Antibody Titer Before and After Conventional Radiotherapy

Studies were conducted to determine the antibody titer of two patients having nasopharyngeal carcinoma (NPC) at various time points with the immunoassay protocol according to the present invention. Both patients received a radiotherapy for NPC which is 2-2.3 Gy/fraction daily for a total dose of 65-70 Gy. In the case of the first patient (“Case 1”), blood samples were collected 1 week prior to the first day of the radiotherapy treatment, as well as 4 and 6 weeks thereafter. For the second patient (“Case 2”), blood samples were collected 1 week prior to the first day of the radiotherapy treatment, as well as 1, 3, and 5 weeks thereafter. The anti-NPC titer was measured with ELISA coated with 8 Gy-irradiated NPC cell lysate as described in Example 12.

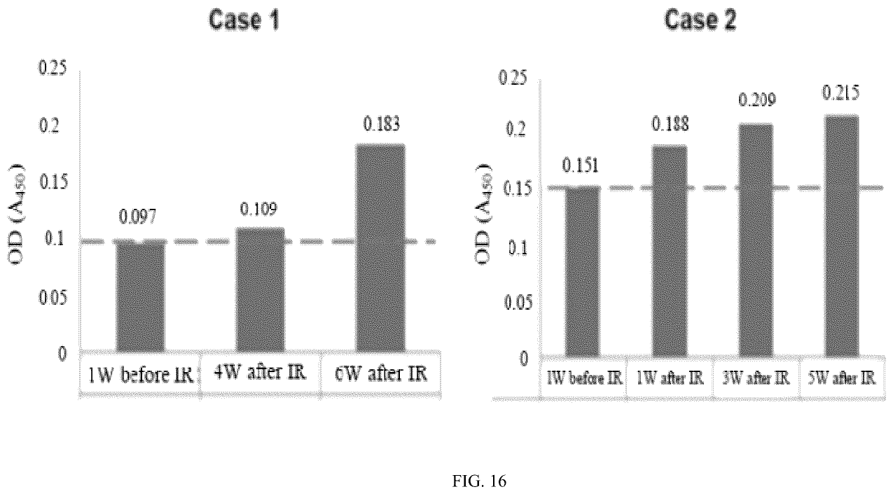

As shown in FIG. 16, both patients exhibited an increase in the anti-NPC antibody by week 4, and a significant increase was observed after around 5-6 weeks. The results demonstrate that the antibody titer can be used as an indicator to monitor a patient's anti-cancer immune response.

### Example 20—Evaluation of Dynamic Changes in Anti-NPC Lymphocyte Transformation and Lymphocyte Subsets after Chemotherapy and Conventional Radiotherapy

Studies utilizing immunoassay protocol according to the present invention were conducted to assess the levels of lymphocyte transformation (LT) and subsets of lymphocytes in 25 patients having NPC before and after Gemcitabine and Cisplatin (GP) induction chemotherapy in combination with radiotherapy. One treatment dose of GP was Gemcitabine at 1000 mg/m² on day 1 and 8 and Cisplatin at 20 mg/m² on day 1 and 3. After a resting period of 3 weeks, additional courses were repeated until the total number of courses reached 4-6. The radiotherapy was 2-2.3 Gy/fraction/day for 5 days a week, reaching a total dose of 65-70 Gy. The assessment of alteration of LT and subsets of lymphocytes were performed as exemplified in FIG. 10 and Examples 11 and 12 with 8 Gy irradiated NPC cell lysate used as a mitogen.

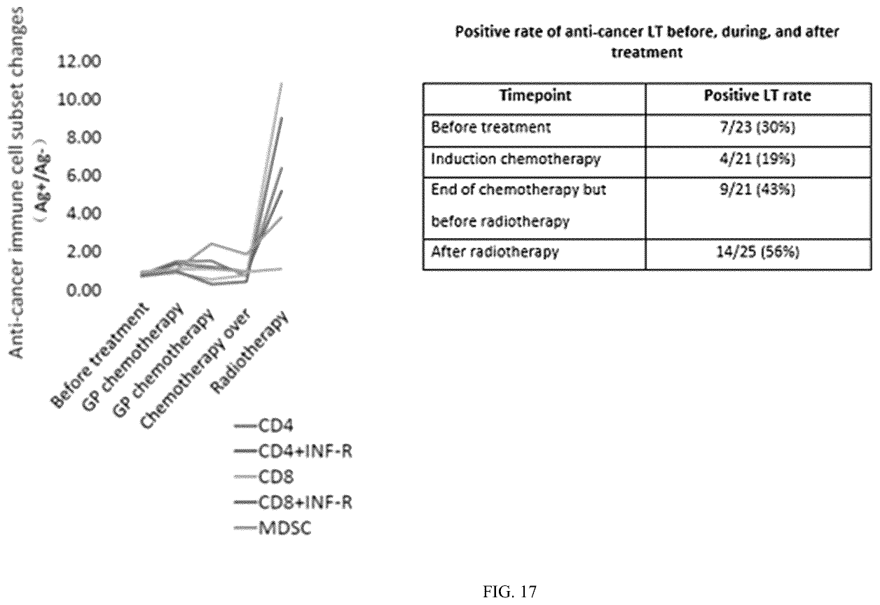

As shown in FIG. 17 on the right side, the LT results were such that, before the treatment, 30% of the patients were positive for LT as an indicator of NPC-induced background immune response. During the chemotherapy, the positive rate of LT decreased to 19%, presumably due to the cytotoxic effects of the chemotherapy on the patients' immune cells. After the chemotherapy the LT rate increased to 43%. The subsequent radiotherapy further increased the positive rate of LT to 56%.

FIG. 17 on the left shows the levels of lymphocyte subsets present in the patients. The results indicate that the levels of subsets of T lymphocytes (i.e., CD₄, CD₄⁺IFNγ⁺, CD₈, CD₈⁺IFNγ⁺, and MDSC) were low before and during the GP treatment, but greatly surged after the radiotherapy. This strongly suggests that the radiotherapy not only induced the formation of an “in situ vaccine,” but also avoided damaging the host immune system, greatly enhancing the anti-cancer immunity of the patients.

The above results taken together indicate that both the cellular and humoral anti-cancer immunities were excited by the radiation-induced “in situ vaccine,” and the immunoassay protocol according to the present invention was useful in assessing the effects of the treatment with in vitro assays using neoantigens as targets or mitogens. Since the immunoassay protocol can be utilized on patients with different cancers, it offers a practical way for oncologists to monitor and assess any changes in a patient's immune response to a given treatment course. The results can be used as a guide to adjust the treatment if necessary.

### Example 21—Experimental Schemes for the Evaluation of the Effects of an Induced Neoantigen Vaccine Prepared In Vitro on Primary Tumor Growth (1)

According to the present invention, a host's own tumor cells can be induced to produce neoantigens in vitro which can be used to immunize to the host to activate the host's anti-tumor immunity. In Examples 21-23, several studies were conducted on mouse models to demonstrate that tumor cells can be induced to produce neoantigens by subjecting the tumor cells to a “survival pressure” in vitro, and that such neoantigens can be used as a vaccine to effectuate an anti-cancer effect on the mice better than vaccines made from uninduced tumor cells.

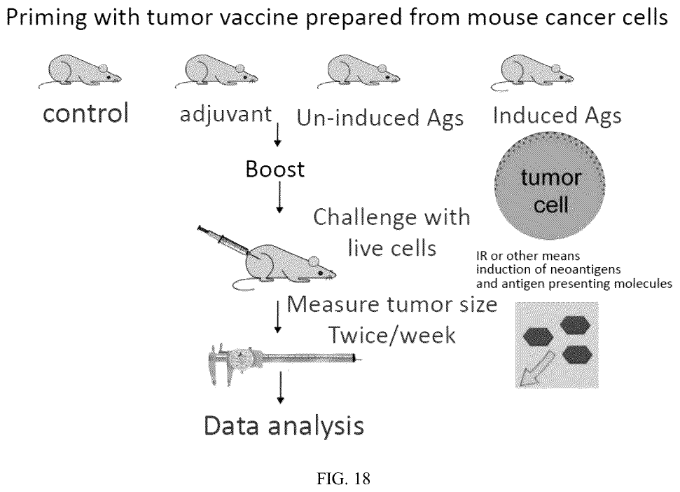

To prepare an induced neoantigen vaccine resembling a “self-tumor” vaccine for purposes these studies, the 4T1-luciferase breast cancer cells were subjected to 8 Gy induction radiation (as “Induced 4T1”) or no induction radiation (as “Uninduced 4T1”). As depicted in FIG. 18, the syngeneic BABL/c mice were divided into 4 groups (6 mice per group): 1) a group that received no immunization (“Control” group); 2) a group that was immunized with Freund's adjuvant alone (“Adjuvant” group); 3) a group that was immunized with 0.5 mg/mouse of Uninduced 4T1 mixed with Freund's adjuvant (“Uninduced Ags” group); and 4) a group that was immunized with 0.5 mg/mouse of Induced 4T1 mixed with Freund's adjuvant (“Induced Ags” group). Immunization protocol was the same for all groups, i.e., the first immunization was given as a primer, followed by three booster immunizations starting 3 weeks after the primer with one-week intervals between the boosters. 3 days after the last booster immunization, 5×10⁶ 4T1-luciferase breast cancer cells were subcutaneously injected into the left and right hind limbs of each mouse, and the tumor size of each mouse was measured twice a week for about 3 weeks.

### Example 22—Evaluation of the Effects of an Induced Neoantigen Vaccine Prepared In Vitro on Primary Tumor Growth (3): Tumor Growth Curve

The tumor growth curves of the mice of Example 21 as shown in FIG. 19 show that the growth of tumors in Induced Ags group was much slower than other groups, including the Uninduced Ags group (P<0.05).

### Example 23—Evaluation of the Effects of an Induced Neoantigen Vaccine Prepared In Vitro on Primary Tumor Growth (4): Tumor Growth Curve

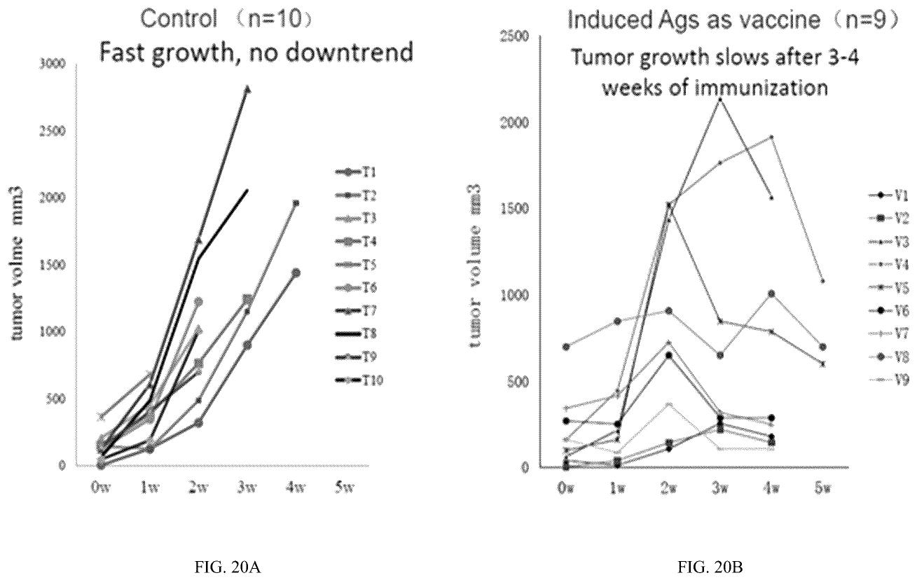

In a study similar to Example 21, 10 mice in a Control group received only a PBS solution and 9 mice in an Induced Ags group received a vaccine derived from 8 Gy radiation-induced neoantigens. The immunization commenced at the same time the 2×10′ tumor cells were subcutaneously injected into the abdomen. Measurements of tumor growth were made and plotted as curves in FIGS. 20A and 20B. The results demonstrate that the tumor size of the Control group increased linearly with no sign of tumor reduction (FIG. 20A), whereas the tumor size of in the Induced Ags group began to show a downward trend within 3-5 weeks after inoculation (FIG. 20B), suggesting that immunization with the vaccine derived from induced neoantigens may take about 3-5 weeks for an anti-cancer effect to manifest itself, which is consistent with a commonly understood pattern of immunity generation.

The results of Examples 21-23 demonstrate that a vaccine made with neoantigens induced by a survival pressure (e.g., 8 Gy induction radiation) is more effective in inducing the host's anti-cancer immunity and thereby inhibiting primary tumor growth than a vaccine made with neoantigens that had not been induced by a survival pressure.

### Example 24—the Effect of an Induced Neoantigen Vaccine Against Metastasis after Immunization

Metastatic control is the key to cancer treatment and thus a study was conducted to demonstrate the effects of an induced neoantigen vaccine according to the present invention on simulated lung metastasis in a mouse model. As described in Example 21, the 4T1-luciferase cells were subjected to 8 Gy induction radiation (as “Induced 4T1”) or no induction radiation (as “Uninduced 4T1”) to obtain antigens as vaccines. The syngeneic BABL/c mice were divided into 3 groups (6 mice/group): 1) a group that was immunized with saline solution alone (“PBS” group); 2) a group that was immunized with 0.5 mg/mouse of Uninduced 4T1 antigens mixed with Freund's adjuvant (“Original” group); and 3) a group that was immunized with 0.5 mg/mouse of Induced 4T1 antigens mixed with Freund's adjuvant (“8 Gy” group). The immunization protocol was the same for all groups, i.e., the first immunization was given as a primer, followed by three times booster immunization starting 3 weeks after the primer with one-week intervals between the boosters.

3 days after the last booster immunization, 2×10⁶ 4T1-luciferase cells were intravenously injected via the tail vein of each mouse. 17 days later, the mice were intraperitoneally injected with 100 μL of 60 mg/mL of luciferin. 20 min later, the mice were anesthetized with isoflurane inhalation anesthesia, and then the extent and the size of lung metastases were imaged with the IVIS Spectrum in vivo.

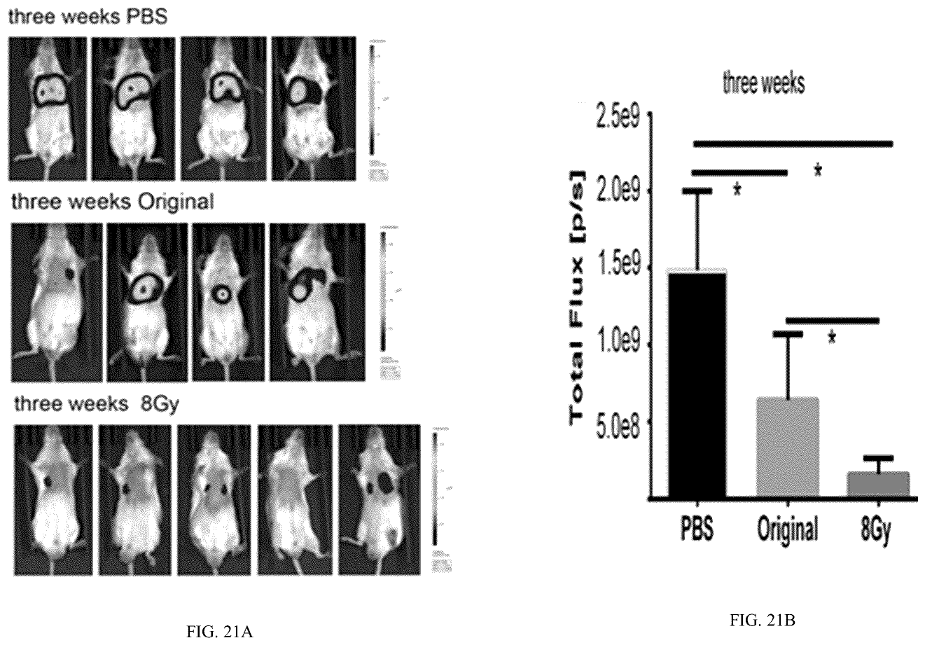

As shown in FIG. 21A, the mice in the 8 Gy group (third row) had much less visible tumors (and thus stronger anti-cancer immunity) than those in the Original group (second row) and PBS group (first row) (P<0.05). FIG. 21B also demonstrates that the total flux measurement for the PBS groups was the greatest, while the 8 Gy group exhibited the least total flux measurement at three weeks. These results demonstrate that the active anti-cancer immunity triggered by immunization of the neoantigen vaccine made under a survival pressure is capable of blocking metastases.

### Example 25—an Induced Neoantigen Vaccine Triggers Anti-Cancer Immunity Analyzed by Various Immunoassay Experiments (1): 8 Gy Induction Radiation Promotes Production of Antigen Presenting Molecules in 4T1 Cells, and Immunization Increased Anti-Cancer Antibodies in Mice

In Examples 25-29, various immunoassay experiments were conducted on 8 Gy-induced 4T1 breast cancer cells and blood samples of mice immunized with lysates of the 8 Gy-induced 4T1 cells to demonstrate the anti-cancer effects of the lysates of the 8 Gy-induced 4T1 cells as an induced neoantigen vaccine. The vaccine was prepared and used to immunize the mice similar to the methods described in Example 21. The results of Examples 25-29, separately and cumulatively, show that both cellular and humoral anti-cancer immunity was activated.

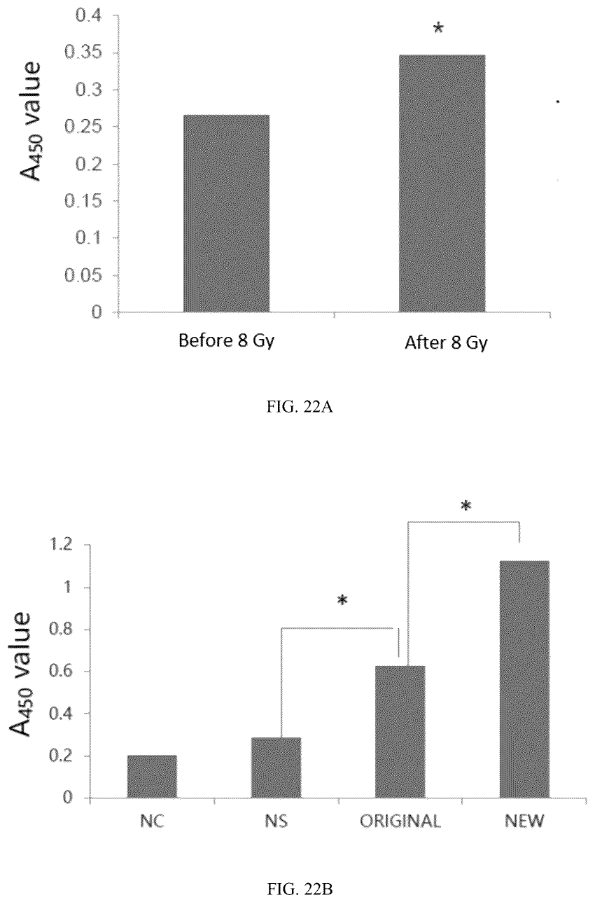

FIG. 22A shows that HSP70, a heat shock protein responsible for neoantigen presentation to the immune system, increased in the lysate of 4T1 breast cancer cells 48 hours after they were irradiated with 8 Gy induction radiation (“After 8 Gy”), compared to the amount of HSP70 present in the cells before such radiation (“Before 8 Gy”). HSP70 was measured with ELISA. The results suggest that radiation-induced neoantigens can be better presented to immune cells because of the increased HSP70.

As shown in FIG. 22B, antibody titer in the plasma samples of four different groups of mice was evaluated. The 4T1 breast cancer cells were irradiated with one-time dose of 8 Gy of induction radiation (“induced 4T1 antigens”) or no radiation (“uninduced 4T1 antigens”) and lysed after 21 days of culture. The four groups of mice were: (1) a group that did not receive any vaccine (“NC”); (2) a group that was immunized with saline (“NS”); (3) a group that was immunized with uninduced 4T1 antigens (“ORIGINAL”); (4) a group that was immunized with induced 4T1 antigens (“NEW”). An ELISA plate was coated with 4 μg/mL lysates of 8 Gy-irradiated 4T1 cells and the plasma samples from each group at a dilution of 1:20000 were added to the coated wells, and the standard procedure for ELISA assay for antibody titer was performed as described in Example 12. FIG. 22B shows the results of the antibody titer in the four groups. The results indicate that the mice immunized with 8 Gy induced 4T1 antigens (i.e., the “NEW” group) had the highest antibody titer compared to the other three groups.

### Example 26—an Induced Neoantigen Vaccine Triggers Anti-Cancer Immunity Analyzed by Various Immunoassay Experiments (2): 8 Gy-Induced 4T1 Antigens Trigger Lymphocyte Transformation

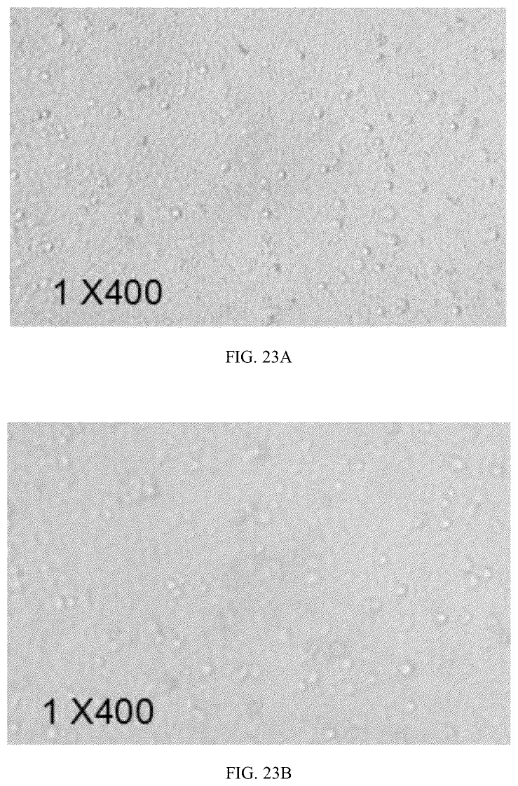

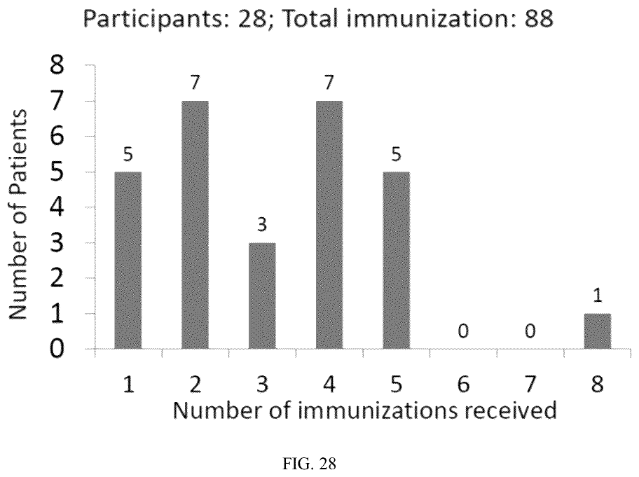

FIGS. 23A and 23B show results of lymphocyte transformation experiments conducted on the lymphocytes of mice immunized with induced 4T1 antigens. Lymphocytes separated from blood of the mice were cultured either in the presence of 4 μg/mL of 8 Gy-induced 4T1 cell lysate as a mitogen (“Ag⁺”) or without such mitogen (“Ag⁻”). Photos of the lymphocytes were taken with a microscope after the second day of culture. FIG. 28 shows that the cells in Ag⁺ group exhibited transformed and proliferating lymphocytes that are bigger (FIG. 23B) compared to the lymphocytes in the Ag group that are smaller and apoptotic (FIG. 23A). The results suggest that the lymphocytes of mice immunized with induced 4T1 antigens can be transformed with radiation-induced 4T1 cell lysate as a mitogen but not without it.

### Example 27—the Induced Neoantigen Vaccine Triggers Anti-Cancer Immunity Analyzed by Various Immunoassay Experiments (3): A Vaccine Comprising 8 Gy-Induced 4T1 Antigens Increases Cancer Cell Death and Antibody Titer

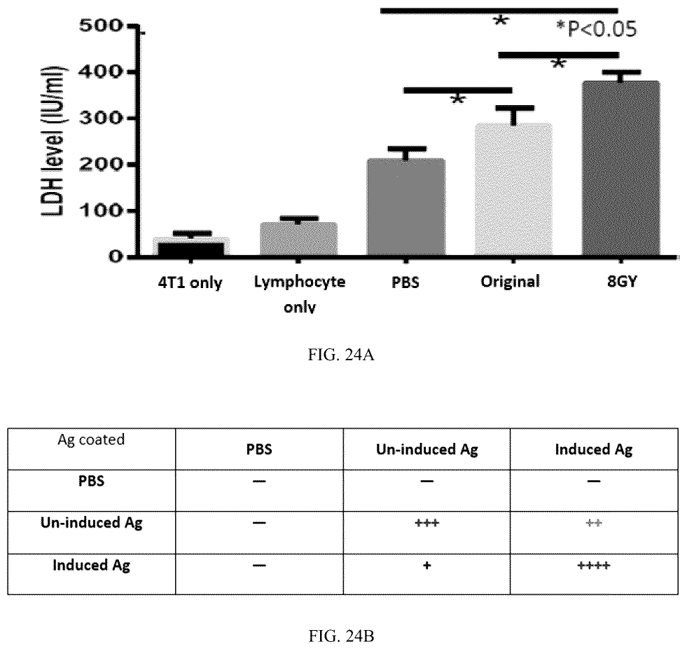

FIG. 24A shows results of a lactate dehydrogenase (LDH) assay to assess the levels of plasma membrane damage in the 4T1 breast cancer cell populations after being exposed to lymphocytes from mice that were immunized with lysates of 8 Gy-induced 4T1 breast cancer cells. LDH assays are generally used as an indicator of cell death/cytotoxicity. The cell culture plate having 24 wells was seeded with 3×10⁴ of cells/well in 1 mL serum-free RPMI 1640 in triplicate. Five groups of cell cultures were prepared:
1) 4T1 breast cancer cells alone (“4T1 only”);
2) lymphocytes from mice obtained 21 days after the mice were immunized with lysates of 8 Gy-induced 4T1 cells (“Lymphocyte only”);
3) 4T1 cells and lymphocytes obtained from mice immunized with PBS control in the respective ratio of 1:10 (“PBS”);
4) 4T1 cells and lymphocytes obtained from mice immunized with lysates of uninduced 4T1 cells in the respective ratio of 1:10 (“Original”); and
5) 4T1 cells and lymphocytes obtained from mice immunized with lysates of 8 Gy-induced 4T1 cells in the respective ratio of 1:10 (“8GY”).

After 6 hours of culture, the media from each well was collected to measure the level of released LDH as a biomarker of dead cells. Generally, the LDH levels correlate with the number of dead cells. The LDH levels of Groups 1 and 2 were low, whereas higher LDH levels (and thus cells deaths) were observed Groups 3 to 5. Of these, Group 5 exhibited the highest level of LDH, indicating more cell deaths than Groups 3 or 4. The results strongly suggest that killing of 4T1 cells can be greatly triggered by immunization with a vaccine derived from 8 Gy-induced 4T1 lysates, much better than uninduced 4T1 lysates.

FIG. 24B demonstrates that humoral immunity induced by various vaccines can be assessed by an antibody titer using ELISA. The plasma samples were collected from four groups of mice: (1) a group that did not receive any vaccine (“normal”); (2) a group that was immunized with a PBS solution (“PBS”); (3) a group that was immunized with uninduced 4T1 antigens (“Uninduced Ag”); and (4) a group that was immunized with induced 4T1 antigens (“Induced Ag”). The ELISA plate was coated with 4 μg/mL lysates of 8 Gy-irradiated 4T1 cells and the plasma samples from each group at a dilution of 1:20000 were added to the coated wells, and the standard procedure for ELISA assay for antibody titer was performed as described in Example 12. The results in FIG. 24B (results for “normal” group not shown) indicate that the mice immunized with the lysates of 8 Gy-irradiated 4T1 cells had the highest anti-cancer neoantigen titer as compared to mice immunized with PBS or uninduced 4T1 antigens.

### Example 28—an Induced Neoantigen Vaccine Triggers Anti-Cancer Immunity Analyzed by Various Immunoassay Experiments (4): A Vaccine Comprising 8 Gy-Induced 4T1 Antigens Increases CD4⁺, CD8⁺ T Cells and Reduces Inhibitory Treg Cells

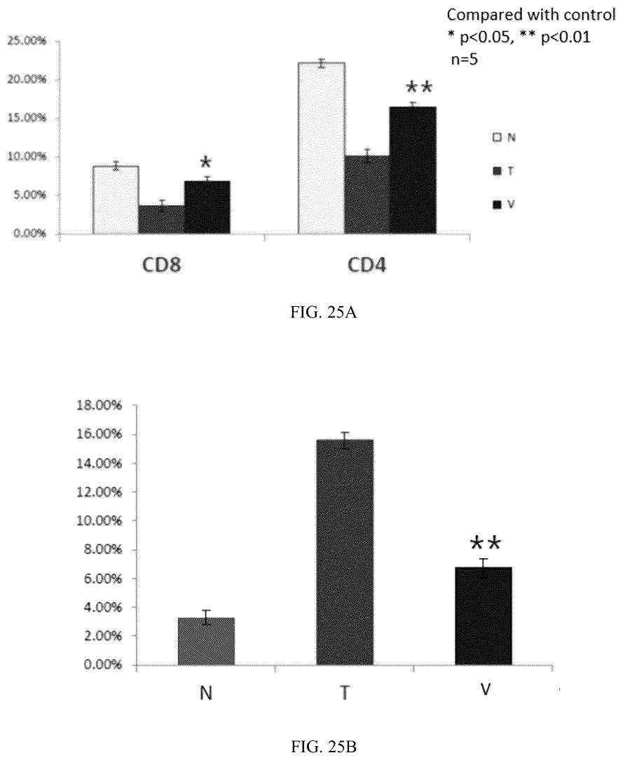

FIG. 25A summarizes percentages of lymphocyte subsets CD4 and CD8 detected in the blood of three groups of mice (5 mice/group): 1) normal mice (Group N); 2) mice immunized with PBS for 28 days and then subcutaneously injected with 1×10⁶ of live 4T1 cells (Group T); and 3) mice immunized with 8 Gy-induced antigens for 28 days and then subcutaneously injected with 1×10⁶ of live 4T1 cells (Group V). One week after 4T1 inoculation, the blood of each mouse was collected, and the lymphocytes were stained with FITC-anti-mouse CD4 or CD8 followed by analysis with C6 flow cytometer (BD Inc., USA). The results in FIG. 25A show that, compared with the mice in Group N, the percentages of CD4 and CD8 subsets in Group T were reduced which might be due to the impairment of immunity caused by the tumor. However, CD4 and CD8 percentages in Group V were higher than Group T, suggesting that immunization with the induced-antigen vaccine enhances the host anti-cancer immunity.

FIG. 25B shows the percentages of Treg, a subset of T cells known to suppress an immune response, in the three groups of mice discussed above (Groups N, T, V). The lymphocytes collected from above 3 groups of mice were stained with anti-mouse Treg and analyzed with C6 flow cytometer. The Treg cells with CD4⁺CD25⁺Foxp3⁺ were reduced in Group V as compared to Group T, indicating the induced-antigen vaccine reduced Treg, the negatively regulating T cells. This suggests indirect enhancement of the anti-cancer immunity.

### Example 29—an Induced Neoantigen Vaccine Triggers Anti-Cancer Immunity Analyzed by Various Immunoassay Experiments (5): A Vaccine Comprising 8 Gy-Induced 4T1 Antigens Reduces Inhibitory MDSCs from Both Blood and Bone Marrow

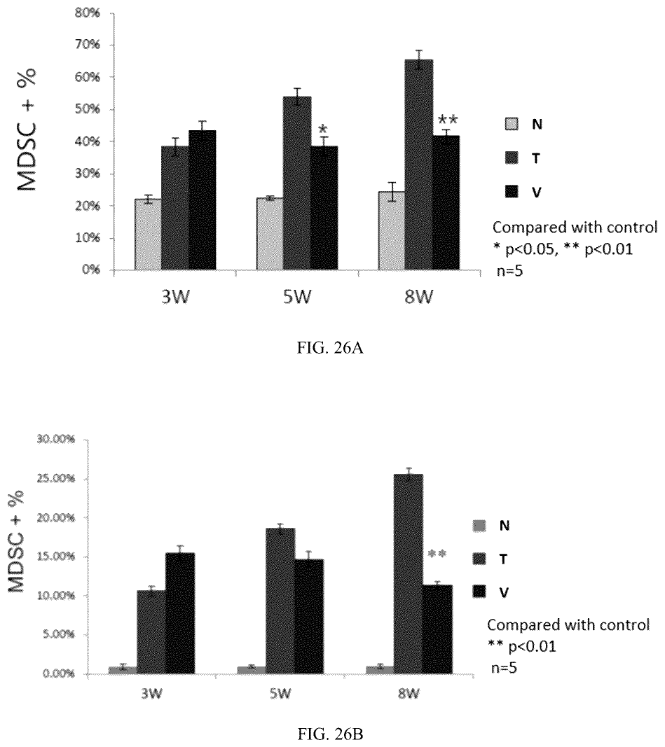

FIGS. 26A-26B show the percentages of myeloid-derived suppressor cells (MDSC) from both the bone marrow and peripheral blood of the three groups of mice described in Example 30 above (Groups N, T, V). MDSC are a subset of negative regulating cells that are generated during an array of pathologic conditions including cancer. The white blood cells (WBC) were collected from the three groups of mice at 3, 5, and 8 weeks after immunization. Live 4T1 cells were subcutaneously inoculated in the abdomen in week 3. The WBCs collected at different time points were stained with FITC-CD11b and PE-Gr-1 and analyzed with C6 flow cytometer. The results showed that the mice bearing the 4T1 tumor (Groups T and V) had increased MDSC levels (CD11b⁺Gr-1⁺) both in the bone marrow (FIG. 26A) and peripheral blood (FIG. 26B) after inoculation at week 3 as compared to normal mice (Group N). While the level of MDSC of tumor-bearing mice in Group T continued to increase with time, the mice in Group V showed decreasing MDSC over time.

All above results of immunological assays using the blood samples from mice immunized with induced-4T1 vaccine indicated that both cellular and humoral anti-tumor immunities were excited and the negative regulators were reduced. The changes in the anti-cancer immunity due to stimulations by a tumor vaccine in vivo can be monitored with in vitro assays with neoantigens as targets or mitogens described in this invention.

### Example 30—Exemplary Flow Chart of the Preparation and Administration of a Self-Tumor Vaccine for Use in Active Immunotherapy

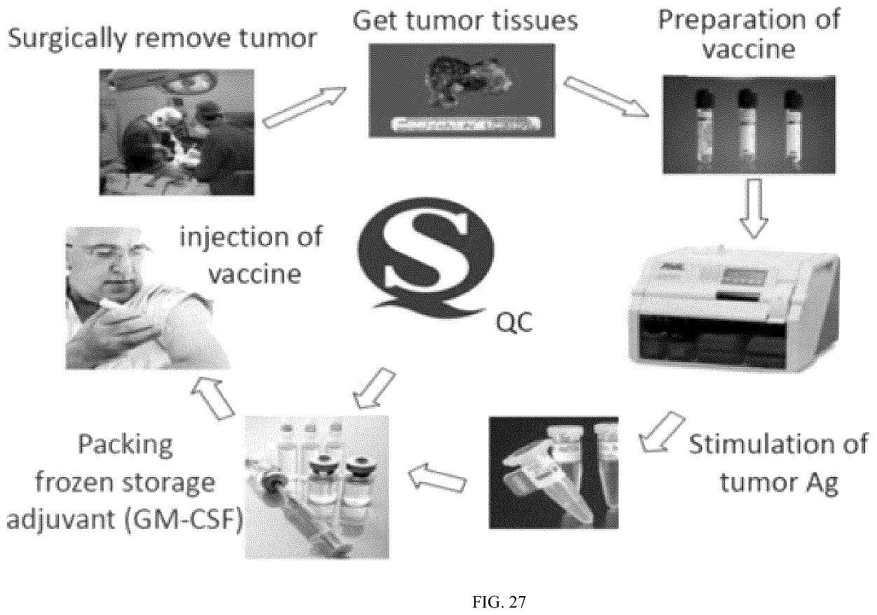

FIG. 27 summarizes an exemplary procedure of preparation and use of individualized tumor vaccine for active immunotherapy.

According to the procedure of FIG. 27, a self-tumor vaccine was prepared. In some embodiments, a tumor tissue of the size of a mung bean, a soybean or a peanut from biopsy or surgery is placed in a sterile 15 mL or 50 mL tube with RPMI 1640 containing 1000 IU/mL of penicillin/streptomycin, then transferred to a GLP (good laboratory practice) lab at 4° C. within 2 hours. Under a super clean cabinet in a sterile condition, the tumor tissues are grinded on iron mesh then filtered with 150-micron nylon mesh to obtain the passing cells. The cells are irradiated with 8 Gy in the cell irradiator according to the present invention and cultured in a 10 cm or 15 cm diameter tissue culture plates with 10-20 mL RPMI 1640 media at 37° C. in a 5% C02 incubator for 2 days to induce the production of neoantigens. The tumor cells are then harvested, centrifuged in 15 mL tubes, resuspended in 10 mL sterile deionized water, and frozen and thawed 5 times. The protein concentration in the cell lysate can be determined by the Bradford protein assay (BCA) method. Thereafter, 0.5 mg of the cell lysate per vial is mixed with 0.5 mL PBS and cryopreserved at −80° C. until it is use as a self-tumor vaccine. Advantageously, the self-tumor vaccine contains a whole set of neoantigens that are induced under a survival pressure and antigen presenting molecules to enhance the presentation of neoantigens to host immune cells. The vaccine can be used on a patient from whom the tumor was derived without the need for an HLA matching. The whole preparation only takes about 3 days, which is an advantageously fast, easy, simple, and cheap preparation of individualized vaccines.

The timing of immunization is dependent on the individual patient's clinical situation and treatment. In some embodiments, administration of the self-tumor vaccine can be concurrent with radiotherapy or targeted drugs, but not cytotoxic drugs. In one embodiment, the self-tumor vaccine is administered in the four limbs via intradermal injection of 0.5 mg vaccine mixed with 1,000,000 IU/mL of GM-CSF as an adjuvant in the ratio of 1:1. In further embodiments, 3 weeks after this priming immunization, booster immunizations can be performed twice at a 3 week-interval and then at a 6-month interval, depending on the patient's disease situation and the amount of vaccine vials available.

### Example 31—Phase I Clinical Trial Demonstrates the Safety of the Self-Tumor Vaccine (1)

To determine the effects of the individualized active immunotherapy as described in Example 30, a phase I clinical trial was conducted after an Institutional Review Board (IRB) approval to assess the safety of the self-tumor vaccine in 28 patients having different cancers, such as hepatoma, breast/ovarian cancer, etc. As shown in FIG. 28, among the 28 patients, 12 patients were immunized 1-2 times, and 16 patients were immunized three or more times depending on their situations, in the total number of 88 immunizations of the self-tumor vaccines according to the present invention.

The data presented in Examples 32-35 indicate that the patient immunized with the self-tumor vaccine that is prepared according the method of the present invention is clinically safe.

### Example 32—Phase I Clinical Trial Demonstrates the Safety of the Self-Tumor Vaccine (2): Fever after Immunization

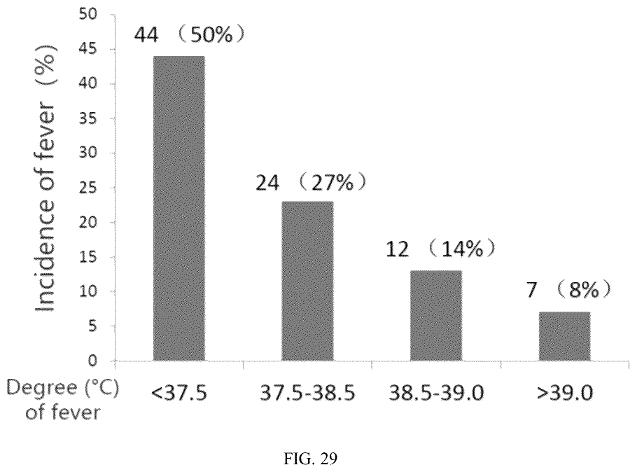

As shown in FIG. 29 the patients of Example 31 were monitored for fever as a sign of systemic side effects. The results show that, while 50% of the subjects had no fever, 41% ran a fever varying from 37.5° C. to 39° C. for 2-6 hours without needing a medicine, 8% had a fever over 39° C. which required physical cooling and chlorpheniramine treatment. Overall, the patients who had a fever began running a fever about 2 hours after injection, and their temperature peaked at 4-10 hours after injection. Temperature regression occurred after 12 to 24 hours after injection.

### Example 33—Phase I Clinical Trial Demonstrates the Safety of the Self-Tumor Vaccine (3): Local Response after Immunization

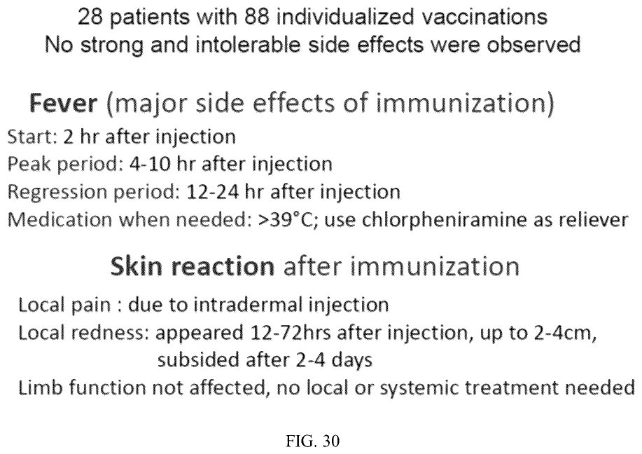

As shown in FIG. 30, local side effects around the injection sites after immunization were pain, redness, and dermatitis-like reaction. Local pain was due to intradermal injection. The local redness appeared 12-72 hours after injection and was up to 2-4 cm in diameter. The dermatitis-like reactions subsided after 2-4 days. Limb function was not affected, and no local or systemic treatment was necessary.

### Example 34—No Autoimmune Antibodies were Produced 7-41 Days after Immunization with a Self-Tumor Vaccine (1)

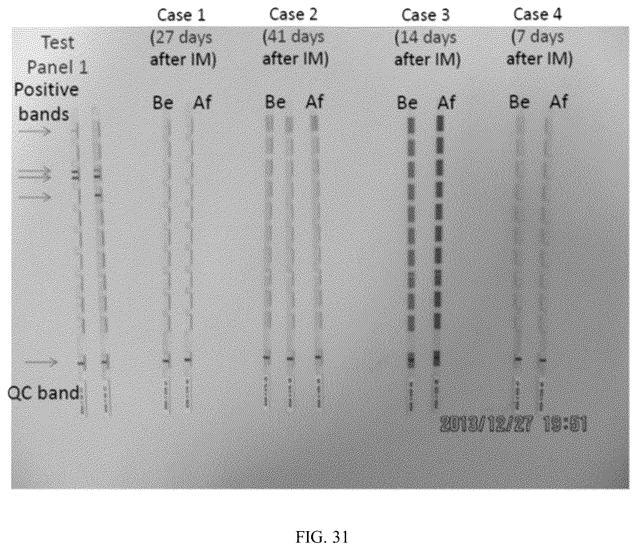

To determine if any antibodies against a patients' self-antigens are generated after immunization with a self-tumor vaccine, Western blotting for self-antigens was performed with capillary electrophoresis on plasma samples collected from before immunization (as background—“Be”) and after immunization (as tests—“Af”) with a panel of different proteins as antigens. The results in FIG. 31 show that the plasma obtained from different patients 7, 14, 27 and 41 days after immunization with the self-tumor vaccines did not contain autoantibodies against self-antigens compared to the patients' own plasma prior to the immunization, suggesting that the self-tumor vaccine did not elicit any autoimmune response.

### Example 35—No Autoimmune Antibodies were Produced 7-41 Days after Immunization with a Self-Tumor Vaccine (2)

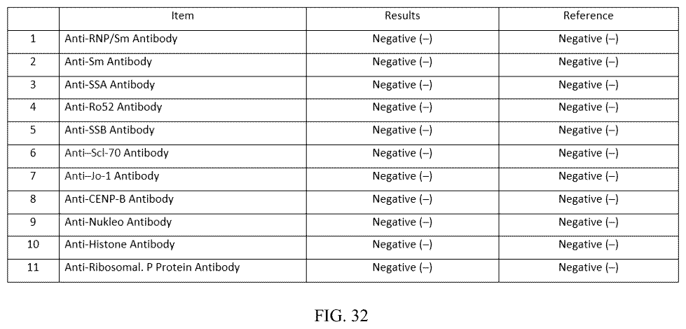

FIG. 32 lists negative results for all of the specific autoantibodies tested, i.e., RNP, Sm, SSA, Ro52, SSB, Scl-70, Jo-1, CENP-B, NUK-leo, Histone, and Ribosomal P Protein.

### Example 36—Effects of Conventional Radiotherapy Combined with Other Therapies on Anti-Cancer Immunity (1): Lymphocyte Transformation

A study was conducted to determine the effects of various treatment methods on anti-cancer immunity in 16 patients with Esophageal cancer. Each patient received one of the following treatments: 1) radiation alone (2.3 Gy/fraction/day for a total dose of 65 Gy (“IR”); 2) radiation in combination with GP chemotherapy (“IR+CR”); and 3) radiation in combination with GM-CSF (100 μg of GM-CSF administered via subcutaneous injection once a week for 4 weeks)(“IR+GM-CSF”). The lymphocyte transformation (LT) assay was performed with blood samples collected before and about 3-10 weeks after the treatment, culturing.

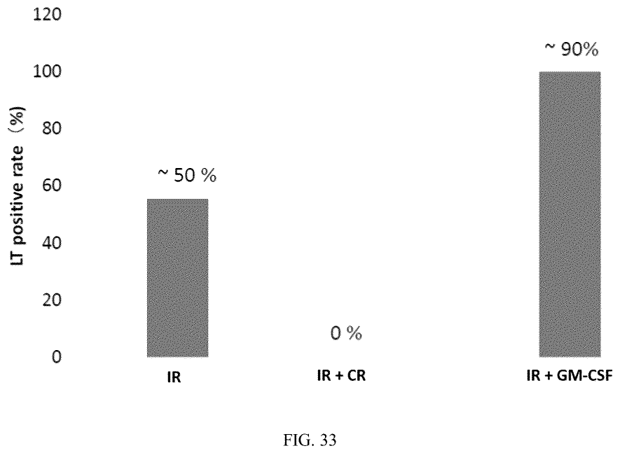

The results in FIG. 33 showed that about 50% (5 out of 9) IR patients and 2 out of 2 IR+GM-CSF patients had an increased LT, while IR+CR patients had no increased LT (0/5). The data strongly suggests that radiation can induce “in situ vaccine” which triggers anti-cancer immunity that is enhanced by GM-CSF but is greatly suppressed by cytotoxic chemotherapy due to its killing of immune cells.

### Example 37—Effects of Conventional Radiotherapy Combined with Other Therapies on Anti-Cancer Immunity (2): GM-CSF Enhanced the Lymphocyte Transformation and Subsets

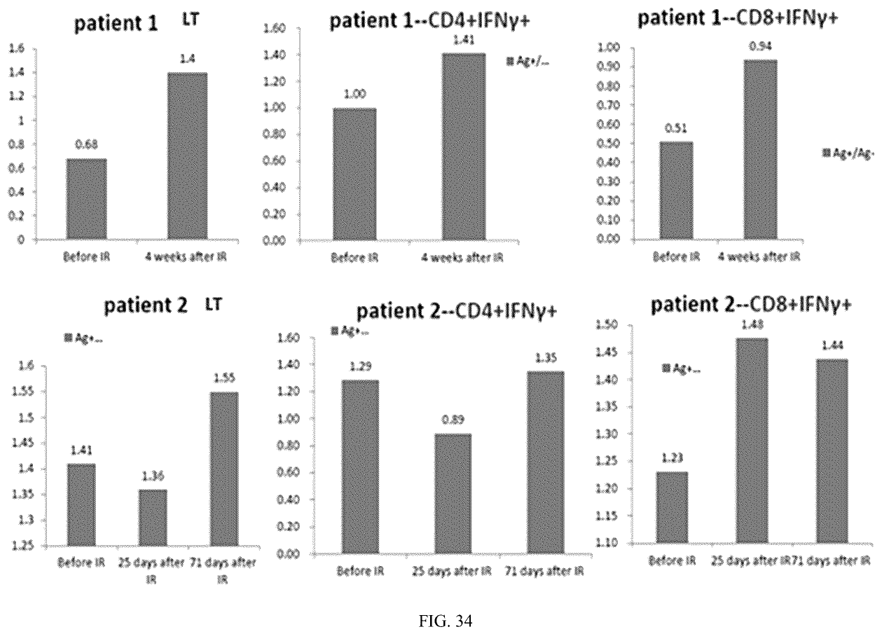

The GM-CSF-enhanced anti-cancer immunity was further studied in the 2 IR+GM-CSF patients by collecting blood samples before and after the IR+GM-CSF treatment. Before the assays, the lymphocytes were cultured with (Ag⁺) or without (Ag⁻) vaccine neoantigens as described above. The results in FIG. 34 show that LT as well as CD4⁺IFNγ⁺ and CD8⁺IFNγ⁺ T cells increased (P<0.05) after 4 or 10 weeks of the treatment, suggesting that different patients have different time patterns of exhibiting their anti-cancer immunity. Thus, a set of immunoassays is important to monitor and evaluate each patient's anti-cancer immunity.

The term “subject” or “patient,” as used herein, describes an organism, including mammals such as primates. Mammalian species that can benefit from the disclosed methods of treatment include, but are not limited to, apes, chimpanzees, orangutans, humans, and monkeys; domesticated animals such as dogs, cats; live stocks such as horses, cattle, pigs, sheep, goats, and chickens; and other animals such as mice, rats, guinea pigs, and hamsters.

As used herein, the singular forms “a,” “an,” and “the” are intended to include the plural forms as well, unless the context clearly indicates otherwise. Further, to the extent that the terms “including,” “includes,” “having,” “has,” “with,” or variants thereof are used in either the detailed description and/or the claims, such terms are intended to be inclusive in a manner similar to the term “comprising.” The transitional terms/phrases (and any grammatical variations thereof), such as “comprising,” “comprises,” and “comprise,” can be used interchangeably.

The transitional term “comprising,” “comprises,” or “comprise” is inclusive or open-ended and does not exclude additional, unrecited elements or method steps. By contrast, the transitional phrase “consisting of” excludes any element, step, or ingredient not specified in the claim. The phrases “consisting” or “consists essentially of” indicate that the claim encompasses embodiments containing the specified materials or steps and those that do not materially affect the basic and novel characteristic(s) of the claim. Use of the term “comprising” contemplates other embodiments that “consist” of or “consisting essentially of” the recited component(s).

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0-20%, 0 to 10%, 0 to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed. In the context of compositions containing amounts of concentrations of ingredients where the term “about” is used, these values include a variation (error range) of 0-10% around the value (X±10%).

I claim:

1. A method of treating cancer in a subject having a cancerous tumor by enhancing anti-cancer active immunity of the subject comprising the step of:
(a) administering to the subject an effective dose of a first induction radiation or a low-dose half-course chemotherapy to induce production of neoantigens by said tumor;
(b) removing at least a portion of the tumor from the subject;
(c) obtaining a vaccine comprising neoantigens produced by cells of the removed tumor after being subjected to a survival pressure in vitro; and
(d) administering said vaccine to the subject.

2. The method according to claim 1, wherein the first induction radiation is utilized for step (a).

3. The method according to claim 2, wherein said first induction radiation is provided in the total dose of 24 Gy to about 32 Gy, which is hypofractionated into about 4 Gy per fraction to about 8 Gy per fraction.

4. The method according to claim 2, wherein the first induction radiation is electromagnetic radiation, particulate radiation, internal radiation therapy, intensity modulated radiation therapy (IMRT), or Stereotactic Body Radiation Therapy (SBRT).

5. The method according to claim 1, wherein step (a) further comprises administering to the subject an immunoenhancer or an immunosuppressive blocker.

6. The method according to claim 1, wherein said survival pressure comprises a second induction radiation of about 6 Gy to about 10 Gy, hypoxia at about 1% oxygen level, or a high temperature at about 39° C. to about 40° C.

7. The method according to claim 1, wherein said vaccine comprises about 1 mg to about 2 mg of said neoantigens, and said vaccine further comprises an adjuvant.

8. The method according to claim 7, wherein the adjuvant is selected from the group consisting of liposomes, cytokines, plant proteins, DNA, inactivated viruses, bacteria, fungi, and microorganisms or their metabolites.

9. The method according to claim 1, further comprising a step of administering an adjuvant to the subject, concurrent with or after step (d), wherein said adjuvant is selected from the group consisting of liposomes, cytokines, plant proteins, DNA, inactivated viruses, bacteria, fungi, and microorganisms or their metabolites.

10. The method according to claim 1, said administration of said vaccine in step (d) is achieved by intradermal injection in all four limbs of the subject.

11. The method of treating cancer according to claim 1, wherein step (d) occurs within about one week of said removal of said tumor if no tumor residue and metastasis are detected in said subject, or after about one month of said removal of said tumor if tumor residue or metastasis is detected.

12. The method according to claim 1, further comprising a step of administering at least one additional dose of said vaccine as a booster immunization after step (d).

13. The method of treating cancer according to claim 1, wherein effects of said method are evaluated by assessing changes in the subject's anti-cancer immunity at least by comparing titers of antibodies and transformation of lymphocytes, said antibodies and lymphocytes obtained from the subject's blood samples collected before and after step (a), wherein said antibodies are detected using first induced neoantigens are obtained from tumor cells that have been subjected to a survival pressure, and
wherein said lymphocyte transformation involves culturing said lymphocytes in the presence of second induced neoantigens as mitogens to enhance lymphocyte proliferation, wherein said second induced neoantigens are obtained from tumor cells that have been subjected to a survival pressure, and said first and second induced neoantigens are same or different.

14. A method of preparing a self-tumor vaccine comprising:
(a) obtaining a tumor tissue from a subject having a cancerous tumor after the subject has been treated with an effective dose of a first induction radiation or a low-dose half-course chemotherapy;
(b) preparing a cell culture from said tumor tissue, wherein said cell culture is subjected to a survival pressure;
(c) obtaining lysates of cells from said cell culture comprising neoantigens;
(d) preparing said self-tumor vaccine comprising said cell lysates.

15. The method according to claim 14, wherein said vaccine does not require human leukocyte antigens (HLA) matching before said vaccine is administered to the subject.

16. An immunoassay protocol comprising:
(a) obtaining a blood sample from a subject having a cancerous tumor;
(b) separating said blood sample into plasma and a fraction comprising lymphocytes;
(b) determining a titer of antibodies in said plasma, wherein said antibodies are detected using first induced neoantigens that are obtained from tumor cells that have been subjected to a survival pressure; and
(c) performing a lymphocyte transformation (LT) test on said lymphocytes, wherein step (c) comprises:
(i) dividing said lymphocytes into two groups;
(ii) culturing a first group of said lymphocytes in the presence of second induced neoantigens as mitogens to enhance proliferation of said lymphocytes and a second group of said lymphocytes in the absence of said mitogen, wherein said second induced neoantigens are obtained from tumor cells that have been subjected to a survival pressure, wherein said first and second induced neoantigens are same or different;
(iii) obtaining measurements of proliferated cells in said first and second groups; and
(iv) determining the extent of lymphocyte proliferation by subtracting the measurements of the second group from the first group.

17. The immunoassay protocol of claim 16, wherein said antibody titer is determined using Enzyme-Linked Immunosorbent Assay (ELISA), and wherein ELISA wells are coated with said first induced neoantigens prior to sample loading.

18. The immunoassay protocol of claim 16, wherein said further comprising a lymphocyte subset test on the first group of lymphocytes, a cytokine assay on said plasma, and/or a CTC test on a whole blood of said blood sample.

19. The immunoassay protocol of claim 16, wherein said blood sample is obtained prior to a cancer treatment, and said steps (a)-(c) are performed on a second blood sample, wherein the results of said antibody titer and said LT test of said blood samples are compared.

20. The method according to claim 1, wherein the survival pressure is in the form of a second induction radiation generated by a cell irradiator comprising:
a high voltage transformer comprising an X-ray tube for creating X-ray radiation;
an X-ray chamber for accommodating said cell sample therein; and
a control system for adjusting the voltage and current of said high voltage transformer, such that said high voltage transformer produces X-ray in the dose rate of about 0.5 Gy/min to about 2 Gy/min and delivers said X-ray radiation to said cell sample in the total dose ranging from about 6 Gy to about 10 Gy in one sitting or in two or more fractions.
